SEQUENCE LISTING (3 sequences)

SEQ ID NO: 1 - DNA, 1311 bp, Homo sapiens
atggaccgcggcacgctccctctggctgttgccctgctgctggccagctgcagcctcagccccacaagtcttgcagaaacagtccattgtgaccttcagcctgtgggccccgagagggacgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcgg

SEQ ID NO: 2 - protein, 437 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQR

SEQ ID NO: 3 - protein, 633 aa, Homo sapien
ETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

(19) United States
(12) Patent Application Publication
(10) Pub. No.: US 2009/0170767 A1
(43) Pub. Date: Jul. 2, 2009

(54) Soluble Endoglin Compounds for the Treatment and Prevention of Cancer

(75) Inventors: S. Ananth Karumanchi, Chestnut Hill, MA (US); Vikas P. Sukhatme, Newton, MA (US)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 12/227,546

(22) PCT Filed: May 31, 2007
(86) PCT No.: PCT/US2007/012828

§ 371 Date: Feb. 27, 2009

Related U.S. Application Data
(60) Provisional application No. 60/809,462, filed on May 31, 2006.

(51) Int. Cl. A61K 38/16; C12N 5/06; C07H 21/04

(52) U.S. Cl. 514/12; 435/375; 536/23.5

(57) ABSTRACT

Disclosed herein are soluble endoglin compounds and kits, pharmaceutical compositions, and articles of manufacture containing soluble endoglin compounds. Also disclosed herein are methods for treating an angiogenesis disorder, such as cancer, using soluble endoglin compounds, provided alone or in combination with a chemotherapeutic agent, an angiogenesis inhibitor, or an antiproliferative compound.

### BACKGROUND OF THE INVENTION

In general, this invention relates to soluble endoglin compounds (e.g., a soluble endoglin protein, or a biologically active fragment, derivative, or analog thereof) and methods of using soluble endoglin compounds for the treatment and diagnosis of various proliferative and angiogenic diseases including cancer.

Transforming growth factor-β (TGF-β) is a multifunctional cytokine originally named for its ability to transform fibroblasts to cells capable of anchorage-independent growth. TGF-β refers to a family of proteins that are primarily produced by hematopoietic and tumor cells and can regulate growth and differentiation of cells from a variety of both normal and neoplastic origins. TGF-β upregulation is known to be involved in a number of pathologic conditions including fibrotic diseases of the lungs, liver, and kidneys; atherosclerosis and arteriosclerosis; viral infections; immunological and inflammatory responses; and proliferative disorders such as cancer.

Studies to date correlating prognosis with TGF-β levels in different cancers have not all been consistent; in some cancers TGF-β is thought to have a growth-suppressing function and in some cancers, TGF-β is upregulated and is thought to have a growth-promoting function. In particular, cancer cells that have acquired mutations in TGF-β signaling pathways appear to be most aggressive or have a metastatic phenotype. It is believed that these acquired mutations allow the cancer cells to escape from the negative growth-suppressive abilities of TGF-β, but still allow the cancer cells to metastasize through the paracrine effects of TGF-β such as pro-angiogenesis and immunomodulatory effects. Recent studies suggest that the pathogenic role of TGF-β seems to correlate with the more malignant or metastatic forms of cancer. For example, TGF-β appears to exert pleiotropic effects in the oncogenesis of breast cancers in a contextual manner. TGF-β functions as a potent growth inhibitor of normal mammary epithelial cells and a number of breast cancer cell lines and can suppress tumorigenesis at an early stage by direct inhibition of angiogenesis and tumor cell growth. However, over-production of TGF-β by an advanced breast tumor may accelerate disease progression through indirect stimulation of angiogenesis and immune suppression.

Endoglin has been shown to be a regulatory component of the TGF-β receptor complex, which modulates angiogenesis, proliferation, differentiation, and apoptosis. In particular, endoglin binds TGF-β1 and TGF-β3 with high affinity and forms heterotrimeric associations with the TGF-β signaling receptors types I and II. Endoglin also binds several other members of the TGF-β superfamily including activin-A, bone morphogenic protein-2 (BMP-2) and BMP-7. The reduction of endoglin levels in human umbilical vein endothelial cells (HUVECs) leads to in vitro angiogenesis inhibition and massive cell mortality in the presence of TGF-β1. Endoglin null mice die in utero with impaired vasculature, indicating the pivotal role of endoglin in vascular development. Endoglin is a homodimeric cell membrane glycoprotein that shares sequence identity with betaglycan, a TGF receptor type III. Mutations in the coding region of the endoglin gene are responsible for haemorrhagic telangiectasia type 1 (HHT1), a dominantly inherited vascular disorder characterized by multisystemic vascular dysplasia and recurrent hemorrhage. A soluble form of endoglin has also been identified and found to be present at increased levels in patients with metastatic breast and colorectal cancer; however, the exact functional role of the soluble endoglin in the pathogenesis of cancer is unclear.

Although TGF-β clearly inhibits the growth and development of early stage tumors, an accumulating body of evidence implicates TGF-β signaling as a stimulus necessary for the metastasis and dissemination of late stage tumors. The ability of TGF-β to induce cancer growth and metastasis suggests that developing therapeutics to antagonize and/or circumvent TGF-β signaling may prove effective in treating cancers, possibly by blocking the pro-angiogenic function of TGF-β.

Thus, there is a pressing need for therapies that control TGF-β signaling to treat or prevent cancer, in particular those cancers that are associated with angiogenesis.

### SUMMARY OF THE INVENTION

We have discovered a novel soluble form of endoglin of placental origin that is present in the sera of pregnant women. We have purified and characterized the circulating soluble endoglin and have demonstrated that it is an N-terminal cleavage product of full-length endoglin. Soluble endoglin may be formed by cleavage of the extracellular portion of the membrane-bound form by proteolytic enzymes, such as metalloproteinases. We have discovered that soluble endoglin interferes with TGF-β1 and TGF-β3 binding to its receptor leading to decreased signaling such as a reduction in Smad2/3-dependent transcription. We have also discovered that soluble endoglin has an anti-angiogenic effect. Finally, we have discovered that soluble endoglin compounds (e.g., a soluble endoglin nucleic acid molecule, soluble endoglin proteins, or biologically active fragments, derivatives, or analogs thereof), can be used to treat or prevent angiogenic or proliferative disorders that are characterized by increased TGF-β activity or expression levels, such as cancer, particularly those cancers that are associated with angiogenic activity or both angiogenic activity and increased TGF-β activity or expression levels. In specific embodiments, the soluble endoglin compound (e.g., a soluble endoglin nucleic acid molecule, a soluble endoglin protein, or a biologically active fragment, derivative, or analog thereof) can be used to treat proliferative diseases, such as cancer, where the angiogenic activity is TGF-β-mediated. Examples of additional disorders that can be treated or prevented by the therapeutic methods of the invention are described in U.S. Patent Application Publication No. 20040131616, herein incorporated by reference.

Accordingly, in a first aspect the invention features a substantially purified soluble endoglin protein, or biologically active fragments, derivatives, or analogs thereof, which have a sequence that is substantially identical to: the amino acid sequence of SEQ ID NO: 2; the amino acid sequence of amino acids 1 to 587 of SEQ ID NO: 3; or amino acids 40 to 406 of SEQ ID NO: 3. The invention also provides a soluble endoglin nucleic acid molecule encoding any of the soluble endoglin proteins. The invention also provides pharmaceutical compositions which contain any of the soluble endoglin nucleic acid molecules, soluble endoglin proteins, or biologically active fragments, derivatives, or analogs thereof, described herein and a pharmaceutically acceptable carrier.

In a second aspect, the invention features a method of inhibiting TGF-β biological activity in a cell, that includes contacting the cell with a soluble endoglin compound (e.g., a soluble endoglin nucleic acid molecule, a soluble endoglin protein, or a biologically active fragment, derivative, or analog thereof) in an amount effective to inhibit the biological activity of TGF-β in the cell. The TGF-β can be any TGF-β family member, desirably TGF-β1 or TGF-β3. The cell can be in vitro or in vivo, for example, in a mammal.

In a third aspect, the invention features a method for treating or preventing cancer in a subject in need thereof, that includes administering to the subject a soluble endoglin compound (e.g., a soluble endoglin nucleic acid molecule, a soluble endoglin protein, or a biologically active fragment, derivative, or analog thereof), wherein the administering is for a time and in an amount sufficient to treat or prevent the cancer. The method can be used to treat or prevent any cancer, including but not limited to, cancer of the breast, prostate, colon, lung, head and neck, liver, kidney, renal system, or endometrium. In preferred embodiments, the cancer has angiogenic activity, increased TGF-β (e.g., TGF-β1 or TGF-β3) levels or biological activity, is metastatic or at risk of becoming metastatic, or any combination thereof. In one example, the cancer is a type of cancer that is known to have increased TGF-β levels.

The method can be used, for example, to treat metastasis or reduce the size or extent of the metastasis in a metastatic cancer, to prevent or reduce the likelihood of metastasis in a subject having a primary cancer that is at risk of becoming metastatic, or as a preventive measure in a subject having an increased risk for metastatic cancer (e.g., a subject having a known BRCA1 or BRCA2 mutation).

Optionally, the method can further include administering to the subject an additional cancer therapy selected from the group consisting of surgery, radiation therapy, chemotherapy, immune therapy (e.g., cytokines, cancer-specific antibodies, interferons, or biologics), differentiating therapy, anti-angiogenic therapy, hormone therapy, or hyperthermia. For such combination methods, the soluble endoglin compound (e.g., a soluble endoglin protein, or a biologically active fragment, derivative, or analog thereof) can be administered before, during, or after the additional cancer therapy. Examples of each of these anti-cancer therapies are known in the art and examples are described herein.

Optionally, the method can also include administering to the subject at least one compound selected from the group consisting of a chemotherapeutic agent, an angiogenesis inhibitor, or an anti-proliferative compound. Examples of angiogenesis inhibitors include an anti-angiogenic antibody (e.g., an antibody that binds VEGF-A or an antibody that binds a VEGF receptor and blocks VEGF binding), avastin, sFlt-1, VEGF-trap, endostatin, angiostatin, restin, tumstatin, TNP-470, 2-methoxyestradiol, thalidomide, a peptide fragment of an anti-angiogenic protein, canstatin, arrestin, a VEGF kinase inhibitor, CPTK787, SFH-1, an anti-angiogenic protein, thrombospondin-1, platelet factor-4, interferon-α, an agent that blocks TIE-1 or TIE-2 signaling, an agent that blocks PIH12 signaling, an agent that blocks an extracellular vascular endothelial (VE) cadherin domain, an antibody that binds to an extracellular VE-cadherin domain, an antibody that blocks TGF-β signaling, tetracycline, penicillamine, vinblastine, cytoxan, edelfosine, tegafur or uracil, curcumin, green tea, genistein, resveratrol, N-acetyl cysteine, captopril, a cyclooxygenase-2 (COX-2) inhibitor, celecoxib, and rofecoxib.

In preferred embodiments of either of the above aspects, the soluble endoglin compound is a soluble endoglin polypeptide or a soluble endoglin nucleic acid molecule. In additional preferred embodiments, the soluble endoglin compound is a soluble endoglin polypeptide, or a biologically active fragment, derivative, or analog thereof, that binds a TGF-β family member (e.g., TGF-β1 or TGF-β3) or a TGF-β receptor.

The biological activity of a soluble endoglin, or a fragment, derivative, or analog thereof, can include any known activity of soluble endoglin such as inhibition of TGF-β binding to a TGF-β receptor, inhibition of angiogenic activity, conversion from a pro-angiogenic state to an anti-angiogenic state, or reversal or inhibition of TGF-β-induced Smad2/3 transcriptional activation.

In a fourth aspect, the invention features a kit for the treatment or prevention of cancer in a subject having, or at risk of developing, a cancer, that includes a soluble endoglin compound (e.g., a soluble endoglin nucleic acid molecule, a soluble endoglin protein, or a biologically active fragment, derivative, or analog thereof) and instructions for the use of the soluble endoglin compound for the treatment or prevention of the cancer.

The kit can be used to treat or prevent any cancer, including but not limited to, cancer of the breast, prostate, colon, lung, head and neck, liver, kidney, renal system, or endometrium. In preferred embodiments, the cancer has angiogenic activity, increased TGF-β (e.g., TGF-β1 or TGF-β3) levels or biological activity, is metastatic or at risk of becoming metastatic, or any combination thereof.

Optionally, the kit can also include at least one additional compound selected from the group consisting of a chemotherapeutic agent, an angiogenesis inhibitor, or an anti-proliferative compound.

Additionally or alternatively, for any of the above methods a compound (e.g., polypeptide, small molecule, antibody, nucleic acid, and mimetic) that increases the level or biological activity of soluble endoglin, or a biologically active fragment, derivative, or analog thereof, can be used.

In a fifth aspect, the invention provides an article of manufacture containing a soluble endoglin compound and a label, wherein the label indicates that the composition is for treating or preventing cancer in a subject having, or at risk or developing, a cancer. The soluble endoglin compound in the article of manufacture may be a soluble endoglin polypeptide, or biologically active fragment thereof, containing a sequence substantially identical to the sequence set forth in SEQ ID NO: 2, amino acids 1 to 587 of SEQ ID NO: 3, or amino acids 40 to 406 of SEQ ID NO: 3. In a preferred embodiment, the soluble endoglin polypeptide, or biologically active fragment thereof, binds a TGF-β family member. In another embodiment, the soluble endoglin compound is a soluble endoglin nucleic acid which contains a sequence that encodes a polypeptide having a sequence substantially identical to SEQ ID NO: 2. In another embodiment, the article of manufacture contains a soluble endoglin nucleic acid containing a sequence substantially identical to SEQ ID NO: 1.

In various embodiments of the article of manufacture, the cancer to be treated or prevented is a metastatic cancer. Optionally, the article of manufacture can include one additional compound selected from the group consisting of a chemotherapeutic agent, an angiogenesis inhibitor, and an anti-proliferative compound. In a preferred embodiment, the article of manufacture contains an additional VEGF inhibitor.

While the detailed description presented herein refers specifically to soluble endoglin, TGF-β1, and TGF-β3 it will be clear to one skilled in the art that the detailed description can also apply to family members, isoforms, and/or variants of soluble endoglin, TGF-β1, and TGF-β3.

For the purpose of the present invention, the following abbreviations and terms are defined below.

By “alteration” is meant a change (i.e., increase or decrease). An “alteration” can refer to a change in the expression levels of a soluble endoglin nucleic acid or polypeptide as detected by standard art known methods such as those described below. As used herein, an alteration includes at least a 10% change in expression levels, preferably at least a 25% change, more preferably at least a 40%, 50%, 60%, 70%, 80%, 90%, 95%, 96%, 97%, 98%, 99%, or greater change in expression levels. “Alteration” can also indicate a change (i.e., increase or decrease) in the biological activity of a soluble endoglin nucleic acid molecule or polypeptide. As used herein, an alteration includes at least a 10% change in biological activity, preferably at least a 25% change, more preferably at least a 40%, 50%, 60%, 70%, 80%, 90%, 95%, 96%, 97%, 98%, 99%, or greater change in biological activity. Examples of biological activity for soluble endoglin polypeptides (or biologically active fragments, derivates, and analogs thereof) are described below.

By “angiogenesis” or “angiogenic activity” is meant the generation of new blood vessels into a tissue or organ. Angiogenesis generally involves endothelial cell proliferation. Under normal physiological conditions, humans or animals undergo angiogenesis only in very specific, restricted situations. For example, angiogenesis is normally observed in wound healing, fetal and embryonal development, and formation of the corpus luteum, endometrium, and placenta. “Anti-angiogenic activity” refers to the capability of a composition to inhibit the growth of blood vessels. The growth of blood vessels is a complex series of events, and includes localized breakdown of the basement membrane lying under the individual endothelial cells, proliferation of those cells, migration of the cells to the location of the future blood vessel, reorganization of the cells to form a new vessel membrane, cessation of endothelial cell proliferation, and incorporation of pericytes and other cells that support the new blood vessel wall. “Anti-angiogenic activity” as used herein includes interruption of any or all of these stages, with the end result that formation of new blood vessels is inhibited.

Anti-angiogenic activity may include, for example, endothelial inhibiting activity, which refers to the capability of a composition to inhibit angiogenesis in general and, for example, to inhibit the growth or migration of bovine capillary endothelial cells in culture in the presence of fibroblast growth factor, angiogenesis-associated factors, or other known growth factors. A “growth factor” is a composition that stimulates the growth, reproduction, or synthetic activity of cells. An “angiogenesis-associated factor” is a factor which either inhibits or promotes angiogenesis. An example of an angiogenesis-associated factor is an angiogenic growth factor, such as basic fibroblastic growth factor (bFGF) or vascular endothelial growth factor (VEGF), which are angiogenesis promoters.

By “angiogenic disorder” is meant a disease associated with excessive or insufficient blood vessel growth, an abnormal blood vessel network, and/or abnormal blood vessel remodeling. For example, insufficient vascular growth can lead to decreased levels of oxygen and nutrients, which are required for cell survival. Angiogenesis, in addition to being critical in metastases formation, also contributes to tumor growth. For any tumors, primary and metastatic, to grow beyond a few millimeters in diameter requires angiogenesis. Examples of angiogenic diseases include, but are not limited to, cancers, solid tumors, blood-born tumors (e.g., leukemias), tumor metastasis, benign tumors (e.g., hemangiomas, acoustic neuromas, neurofibromas, trachomas, and pyogenic granulomas), rheumatoid arthritis, psoriasis, ocular angiogenic diseases (e.g., diabetic retinopathy, retinopathy of prematurity, macular degeneration, corneal graft rejection, neovascular glaucoma, retrolental fibroplasia, rubeosis), Osler-Webber Syndrome, myocardial angiogenesis, plaque neovascularization, telangiectasia, hemophiliac joints, angiofibroma, and wound granulation.

By “binding” is meant a non-covalent or a covalent interaction, preferably non-covalent, that holds two molecules together. For example, two such molecules could be a ligand and its receptor, an enzyme and an inhibitor of that enzyme, an enzyme and its substrate, or an antibody and an antigen. Non-covalent interactions include, but are not limited to, hydrogen bonding, ionic interactions among charged groups, van der Waals interactions, and hydrophobic interactions among non-polar groups. One or more of these interactions can mediate the binding of two molecules to each other. Binding may exhibit discriminatory properties such as specificity or selectivity.

By “chemotherapy” is meant the use of a chemical agent to destroy a cancer cell, or to slow, arrest, or reverse the growth of a cancer cell.

By “chemotherapeutic agent” is meant a chemical that may be used to destroy a cancer cell, or to slow, arrest, or reverse the growth of a cancer cell. Chemotherapeutic agents include, without limitation, asparaginase, bleomycin, busulfan carmustine (commonly referred to as BCNU), chlorambucil, cladribine (commonly referred to as 2-CdA), CPT11, cyclophosphamide, cytarabine (commonly referred to as Ara-C), dacarbazine, daunorubicin, dexamethasone, doxorubicin (commonly referred to as Adriamycin), etoposide, fludarabine, 5-fluorouracil (commonly referred to as 5FU), hydroxyurea, idarubicin, ifosfamide, interferon-γ (native or recombinant), levamisole, lomustine (commonly referred to as CCNU), mechlorethamine (commonly referred to as nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, and vincristine.

By “compound” is meant any small molecule chemical compound, antibody (or antigen-binding fragments thereof), nucleic acid molecule, nucleopeptide, or polypeptide, or fragments thereof.

By “differentiating therapy” is meant the treatment of malignant cells in which the malignant cells are treated so that they can resume the process of maturation and differentiation into mature cells. Examples of a therapeutic agents used in differentiating therapy include, but are not limited to all-trans retinoic acid (ATRA), proliferator-activated receptor (PPARγ) agonists (e.g., troglitazone and ciglitazone), and retinoid X receptor (RXR) ligands, (e.g., 9-cis retinoic acid). Other examples of therapeutic agents used in differentiating therapy are known in the art.

By “endoglin” or “Eng,” also known as CD105, is meant a mammalian growth factor that has endoglin biological activity (see, Fonsatti et al., Oncogene 22:6557-6563, 2003; Fonsatti et al., Curr. Cancer Drug Targets 3:427-432, 2003) and is homologous to the protein defined by any of the following GenBank accession numbers: AAH29080 and NP_{—}031958 (mouse); AAS67893 (rat); NP_{—}000109, P17813, VSP_{—}004233, and CAA80673 (pig); CAA50891 and AAC63386 (human); or those described in U.S. Pat. No. 6,562,957. Endoglin is a homodimeric cell membrane glycoprotein which is expressed at high levels in proliferating vascular cells and syncytiotrophoblasts from placentas. There are two distinct isoforms of endoglin, L and S, which differ in their cytoplasmic tails by 47 amino acids. Both isoforms are included in the term endoglin as used herein. Endoglin is an auxiliary component of the TGF-β receptor system, able to associate with the signaling receptor types I (TGF-β receptor-1; TβRI) and II (TGF-β receptor-2; TβRII) in the presence of ligand and to modulate the cellular responses to TGF-β. Endoglin binds to TGF-β family members and, in the presence of TGF-β, endoglin can associate with TβRI and TβRII, and potentiate the response to the growth factors. Endoglin biological activities include binding to TGF-β family members such as activin-A, BMP-2, BMP-7, TGF-β1 and TGF-β3; binding to TGF-β receptors (e.g., TβRI and TβRII); induction of angiogenesis, regulation of cell proliferation, attachment, migration, invasion; and activation of endothelial cells. Assays for endoglin biological activities are known in the art and include ligand binding assays or Scatchard plot analysis; BrdU labeling, cell counting experiments, or quantitative assays for DNA synthesis such as ³H-thymidine incorporation used to measure cell proliferation; and angiogenesis assays such as those described herein or in McCarty et al., Intl. J. Oncol. 21:5-10, 2002; Akhtar et al., Clin. Chem. 49:32-40, 2003; and Yamashita et al., J. Biol. Chem. 269:1995-2001, 1994.

By “soluble endoglin polypeptide” or “sEng” is meant any circulating, non-membrane bound form of endoglin which includes at least a part of the extracellular portion of the endoglin protein and is substantially identical (e.g., at least 60%, 70%, 80%, 90%, 95%, 96%, 97%, 98%, 99%, or 100% identical) to the amino acid sequence encoding the extracellular portion of the endoglin protein (see FIGS. 1 and 3B). Soluble endoglin can result from the cleavage of the membrane-bound form of endoglin by a proteolytic enzyme. One potential cleavage site is at amino acid 437 of human endoglin producing a soluble endoglin polypeptide that includes amino acids 1-437 of the endoglin polypeptide (see, FIGS. 3A and 3B), or a protein that is substantially identical to amino acids 1-437 of the endoglin polypeptide. Additional forms of soluble endoglin of the invention include a protein substantially identical to amino acids 40 (glycine) to 406 (arginine), amino acids 26 (glutamate) to 437 (arginine), amino acids 26 (glutamate) to 587 (leucine) of the human endoglin shown in FIG. 4B; a protein substantially identical to amino acids 1 to 587 of human endoglin (commercially available from R&D Systems, catalog number 1097-EN); any polypeptide that includes one or more of the peptides identified in bold and underlined in FIG. 4B: amino acids 40 (glycine) to 86 (lysine); amino acids 144 (threonine) to 199 (arginine); amino acids 206 (glycine) to 222 (arginine); amino acids 289 (glycine) to 304 (arginine); amino acids 375 (glutamate) to 381 (lysine); and any polypeptide that includes the regions or domains of soluble endoglin that are required for binding to TGF-β (e.g., TGF-β1 and TGF-β3) or TGF-β receptors (e.g., TβRI and TβRII). It should be noted that the numbering of both endoglin and soluble endoglin depends on whether the leader peptide sequence is included. The numbering of endoglin used herein is shown in FIG. 4B, starting at amino acid 26 (where the absent leader peptide sequence would be amino acids 1-25). Soluble endoglin can also include circulating degradation products or fragments that result from enzymatic cleavage of endoglin and that maintain soluble endoglin biological activity. Preferred soluble endoglin polypeptides have soluble endoglin biological activity such as binding to substrates such as TGF-β family members (e.g., TGF-β1 and TGF-β3) or TGF-β receptors (e.g., TβRI and TβRII) or reversing or inhibiting angiogenesis by at least 10%, 20%, 30%, 40%, 50%, 60%, 70%, 80%, 90%, or more. Examples of assays for measuring these activities are known in the art and described in U.S. Patent Application Publication Nos. 20060067937 and 20050267021, and PCT Publication No. WO 06/034507, incorporated herein by reference. For example, soluble endoglin biological activity can include the ability to reverse, reduce, or inhibit angiogenesis induced by TGF-β or the ability to reverse activation of Smad 2/3 or Smad 2/3-dependent transcriptional activation. Soluble endoglin polypeptides may be isolated from a variety of sources, such as from mammalian tissue or cells (e.g., placental tissue or cells), or prepared by recombinant or synthetic methods. The term soluble endoglin also encompasses modifications to the polypeptide, fragments, derivatives, analogs, and variants of the endoglin polypeptide, examples of which are described below.

“Soluble endoglin compounds” include any soluble endoglin polypeptide, nucleic acid molecule, non-peptidyl small molecule chemical compound, peptidyl mimetic, or fragments, derivatives, analogs, or homologs of soluble endoglin. Preferred soluble endoglin compounds have soluble endoglin biological activity (e.g., at least 25%, 30%, 40%, 50%, 60%, 70%, 80%, 90%, 95%, 100%, 120%, 150%, 200%, 250%, or more as compared to a wild type soluble endoglin polypeptide in the same assay).

By “endoglin nucleic acid” is meant a nucleic acid that is substantially identical to a nucleic acid encoding any of the endoglin or soluble endoglin proteins described above. For example, the gene for human endoglin consists of 14 exons, where exon 1 encodes the signal peptide sequence, exons 2-12 encode the extracellular domain, exon 13 encodes the transmembrane domain, and exon 14 encodes C-terminal cytoplasmic domain (see FIGS. 1, 3A, and 3B). Desirably, the endoglin nucleic acid is substantially identical (e.g., at least 60%, 65%, 70%, 75%, 80%, 85%, 90%, 95%, 96%, 97%, 98%, 99%, or 100% identical) to the nucleic acid sequence set forth in FIG. 3A.

By “expression” is meant the detection of a gene or polypeptide by standard art known methods. For example, DNA expression is often detected by Southern blotting or polymerase chain reaction (PCR) and RNA expression is often detected by Northern blotting, RT-PCR, gene array technology, or RNAse protection assays. Methods to measure protein expression levels generally include, but are not limited to Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), and flow cytometry, as well as assays based on a property of the protein including but not limited to enzymatic activity or interaction with other protein partners. Exemplary assays are described in detail in U.S. Patent Application Publication No. 20060067937 and PCT Publication No. WO 06/034507. Any compound that increases soluble endoglin expression levels by at least 10%, 20%, preferably 30%, more preferably at least 40% or 50%, and most preferably at least 60%, 70%, 80%, or 90% or more is considered a therapeutic compound of the invention.

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain at least 10, 20, 30, 40, 50, 60, 70, 80, 90, or at least 100, 200, 300, 400, 500, 600, 700, 800, 900, 1000, 1250, 1500, 1750, 1800 or more nucleotides or at least 10, 20, 30, 40, 50, 60, 70, 80, 90, 100, 150, 200, 250, 300, 350, 400, 450, 500, 550, 600, 640 amino acids or more. Preferred fragments of soluble endoglin will have soluble endoglin biological activity (e.g., binding to TGF-β or TGF-β receptor) and may include, for example, the TGF-β or TGF-β receptor binding domain.

By “heterologous” is meant any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By “homologous” is meant any gene or polypeptide sequence that bears at least 30% homology, more preferably at least 40%, 50%, 60%, 70%, 80%, and most preferably at least 90%, 95%, 96%, 97%, 98%, 99%, or more homology to a known gene or polypeptide sequence over the length of the comparison sequence. A “homologous” polypeptide can also have at least one biological activity of the comparison polypeptide. For polypeptides, the length of comparison sequences will generally be at least 10, 20, 30, 40, 50, 60, 70, 80, 90, 100, 150, 200, 250, 300, 350, 400, 450, 500, 550, 600, 640 amino acids or more. For nucleic acids, the length of comparison sequences will generally be at least 10, 20, 30, 40, 50, 60, 70, 80, 90, or at least 100, 200, 300, 400, 500, 600, 700, 800, 900, 1000, 1250, 1500, 1750, 1800 or more nucleotides. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein or polypeptide.

By “immune therapy” is meant the use of immunological cells or immunological cell products (e.g., cytokines, cancer-specific antibodies, or interferons) to treat cancer in a subject. The immunological cell products may be naturally produced in an animal or may be recombinantly-produced or synthesized using techniques known in the art. The term “immune therapy” also includes the use of biologics to treat cancer.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

By “metastatic disease,” “metastases,” and “metastatic lesion” are meant a group of cells which have migrated to a site distant relative to the primary tumor. “Non-metastatic” refers to tumor cells, e.g., human cancer cells, that are unable to establish secondary tumor lesions distant to the primary tumor. Although not often the case, metastatic disease can occur when no primary tumor has been detected. The cells in a metastatic tumor resemble those in the primary tumor. Metastasis or metastatic disease can be diagnosed in a variety of ways that are known in the art. Generally, metastatic disease is diagnosed using radiological methods such as X-ray, computed tomography (CT) scan, ultrasound, or magnetic resonance imaging (MRI). Positron emission tomography (PET) scan can also be used. Additional techniques such as Circulating Tumor Cell analysis (CTC) can be used to determine the number of epithelial cells present in a sample of bodily fluid (e.g., blood). For example, in normal patients there are very few if any (typically less than 1) epithelial cells/ml of blood. If a patient is found to have a relatively higher CTC count (e.g., at least 2, 3, 5, 10, 15, 20, 25, 50, 100, 250, 500, 1000, or more epithelial cells), this is considered an indicator of metastatic disease and the disease can then be confirmed using additional methods described herein. Such CTC kits are commercially available and include CellSearch™ Epithelial Cell Kit and CellSpotter™ (Veridex, Warren, N.J.). If needed, a biopsy can be performed, either in conjunction with the radiological methods or separately, and the tissue can be examined for molecular markers of the metastatic disease either at the protein, DNA, or RNA level. In a biopsy, metastases are typically diagnosed by the presence of cells, or molecular markers, that are not normally found in the part of the body from which the tissue sample was taken. For example, if a tissue sample taken from a tumor in the lung contains cells that look like breast cells, the doctor determines that the lung tumor is a secondary tumor to the primary breast cancer. The molecular markers can be markers of cancer or metastatic disease (e.g., p53, VHL, or BRCA mutations), markers of the primary tumor, or markers of the primary tumor cell type (e.g., breast cells found in the lung in the above example) or any combination of these. Identification of a metastasis and determination can include the use of several techniques, such as immunohistochemistry, FISH (fluorescent in situ hybridization), gene array profiling, RNA analysis by RT-PCR, and others. It should be noted that metastases may not have an identical profile to the cells of the primary tumor but will have a profile that is substantially more similar to the profile of the primary tumor than to the cells at the metastatic site in question. For example, if a lung biopsy is obtained and analyzed by gene expression profiling, the profile may be 90% identical to the profile obtained from the breast cancer biopsy and only 50% identical to the profile of a lung cell taken from the area surrounding the metastatic site.

By “modulate expression or activity” is meant to either increase or decrease expression or activity, for example, of a protein or nucleic acid sequence, relative to control conditions. The modulation in expression or activity is preferably an increase or decrease of at least 20, 40, 50, 75, 90, 100, 200, 500, or even 1000%. In various embodiments, transcription, translation, mRNA or protein stability, or the binding of the mRNA or protein to other molecules in vivo is modulated by the therapy. The level of mRNA may be determined by standard Northern blot analysis, and the level of protein may be determined by standard Western blot analysis, such as the analyses described herein or those described by, for example, Ausubel et al. (Current Protocols in Molecular Biology, John Wiley & Sons, New York, 2000).

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “preventing” is meant prophylactic treatment of a subject who is not yet ill, but who is susceptible to, or otherwise at risk of, developing a particular disease. Preferably a subject is determined to be at risk of developing cancer or cancer metastasis using diagnostic methods known in the art. For example, when used with relation to metastatic disease, “preventing” can refer to the preclusion of metastatic disease occurrence in a patient diagnosed with a primary cancer. In one example, the preventive measures are used to prevent a primary cancer, that is invasive or prone to metastatic disease, from metastasizing, where the cancer would otherwise be predicted, based on statistic or clinical characteristics of the cancer that are known to be associated with metastatic disease, to metastasize.

By “primary tumor” or “primary cancer” is meant the original cancer and not a metastatic lesion located in another tissue or organ in the subject's body.

By “proliferation” is meant an increase in cell number, i.e., by mitosis of the cells.

By “protein,” “polypeptide,” or “peptide” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “purified” or “substantially pure” is meant separated from other components that naturally accompany it. Typically, a compound (e.g., nucleic acid, polypeptide, small molecule) is substantially pure when it is at least 50%, by weight, free from proteins, flanking nucleic acids, antibodies, and naturally-occurring organic molecules with which it is naturally associated. Preferably, the factor is at least 75%, more preferably, at least 80%, 85%, or 90%, and most preferably, at least 95% or 99%, by weight, pure. A substantially pure factor may be obtained by chemical synthesis, separation of the factor from natural sources, or production of the factor in a recombinant host cell that does not naturally produce the factor. Proteins and small molecules may be purified by one skilled in the art using standard techniques such as those described by Ausubel et al. (Current Protocols in Molecular Biology, John Wiley & Sons, New York, 2000). The factor is preferably at least 2, 5, or 10 times as pure as the starting material, as measured using polyacrylamide gel electrophoresis, column chromatography, optical density, HPLC analysis, or Western blot analysis (Ausubel et al., supra). Preferred methods of purification include immunoprecipitation, column chromatography such as immunoaffinity chromatography, magnetic bead immunoaffinity purification, and panning with a plate-bound antibody.

By “radiation therapy” is meant the use of directed gamma-rays or beta-rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease preferably of 20%, 30% or greater, more preferably of 40%, 50%, 60% or greater, and most preferably of 70%, 75%, 80%, 85%, 90%, 95%, or greater. For example, in some embodiments of the invention, reduce or inhibit can refer to the symptoms of the disorder being treated, the presence or size of metastases, the size of the primary tumor, or the biological activity of a TGF-β family member.

By “sample” is meant a bodily fluid (e.g., urine, blood, serum, plasma, or cerebrospinal fluid), tissue, or cell.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., an endoglin or soluble endoglin sequence. “Substantial identity” may be used to refer to various types and lengths of sequence, such as fill-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman J. Mol. Biol. 147:195-7, 1981); “BestFit” (Smith and Waterman, Advances in Applied Mathematics, 482-489, 1981) as incorporated into GeneMatcher Plus™, Schwarz and Dayhof “Atlas of Protein Sequence and Structure,” Dayhof, M. O., Ed., pp 353-358, 1979; BLAST program (Basic Local Alignment Search Tool; Altschul et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10, 20, 30, 40, 50, 60, 70, 80, 90, 100, 150, 200, 250, 300, 350, 400, 450, 500, 550, 600, 640 amino acids or more. For nucleic acids, the length of comparison sequences will 20 generally be at least 10, 20, 30, 40, 50, 60, 70, 80, 90, or at least 100, 200, 300, 400, 500, 600, 700, 800, 900, 1000, 1250, 1500, 1750, 1800 or more nucleotides. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “surgery” is meant the physical removal of malignant or benign tumor cells from a subject. By the term “surgery” is also meant the removal of a primary tumor or a secondary or metastatic tumor from a patient.

By “transforming growth factor β (TGF-β)” is meant a mammalian growth factor that has TGF-β biological activity and is a member of a family of structurally related paracrine polypeptides found ubiquitously in vertebrates, and prototypic of a large family of metazoan growth, differentiation, and morphogenesis factors (see, for review, Massaque et al., Ann. Rev. Cell Biol. 6:597-641,1990; Massaque et al., Trends Cell Biol. 4:172-178, 1994; Kingsley, Gene Dev. 8:133-146, 1994; and Sporn et al., J. Cell. Biol. 119:1017-1021, 1992). As described in Kingsley, supra, the TGF-β superfamily has at least 25 members, and can be grouped into distinct subfamilies with highly related sequences. The most obvious subfamilies include the following: the TGF-β subfamily, which comprises at least four genes that are much more similar to TGF-β1 than to other members of the TGF-β superfamily; the bone morphogenetic protein family (BMP) the activin subfamily, comprising homo- or hetero-dimers or two subunits, inhibinβ-A and inhibinβ-B. The decapentaplegic subfamily, which includes the mammalian factors bone morphogenic protein-2 (BMP-2) and bone morphogenic protein-4 (BMP-4), which can induce the formation of ectopic bone and cartilage when implanted under the skin or into muscles. The 60A subfamily, which includes a number of mammalian homologs, with osteoinductive activity, including BMP-5, BMP-6, BMP-7, and BMP-8. Other members of the TGF-β superfamily include the gross differentiation factor 1 (GDF-1), GDF-3VGR-2, dorsalin, nodal, mullerian-inhibiting substance (MIS), and glial-derived neurotrophic growth factor (GDNF). It is noted that the DPP and 60A sub-families are related more closely to one another than to other members of the TGF-β superfamily, and have often been grouped together as part of a larger collection of molecules called DVR (dpp and vgl related). Unless evidenced from the context in which it is used, the term TGF-β as used throughout this specification will be understood to generally refer to members of the TGF-β superfamily as appropriate (Massague et al., Annu. Rev. Biochem. 67:753-91, 1998; Josso et al., Curr. Op. Gen. Dev., 7:371-377, 1997). TGF-β functions to regulate growth, differentiation, motility, tissue remodeling, neurogenesis, wound repair, apoptosis, and angiogenesis in many cell types. TGF-β can inhibit or promote cell proliferation, depending on the cell type and the environment, and can stimulate the synthesis of matrix proteins. Additional details regarding TGF-β family members and functions can be found in U.S. Patent Application Publication Nos. 20050276802, 20050267021, and 20040131616, herein incorporated by reference. Preferably, the methods of the invention relate to TGF-β1 and TGF-β3, but can include any of the TGF-β family members described above.

By “treating” is meant administering a compound or a pharmaceutical composition for prophylactic and/or therapeutic purposes or administering treatment to a subject already suffering from a disease to improve the subject's condition or to a subject who is at risk of developing a disease. Treating can include administering a therapy directly to the subject or using an ex vivo approach to therapy. By “treating cancer” or “treating a metastatic disease” is meant that the disease and the symptoms associated with the disease are alleviated, reduced, cured, or placed in a state of remission. More specifically, when soluble endoglin compound (e.g., a soluble endoglin protein, or a biologically active fragment, derivative, or analog thereof) are used to treat a subject with a tumor, it is generally provided in a therapeutically effective amount to achieve any one or more of the following: inhibit tumor growth, reduce tumor mass, or reduce tumor size or presence such that there is no detectable disease, and slow or prevent an increase in the size of a tumor (as assessed by e.g., radiological imaging, biological fluid analysis, cytogenetics, fluorescence in situ hybridization (FISH), immunocytochemistry, colony assays, multiparameter flow cytometry, or polymerase chain reaction). For example, a therapeutic amount can cause a qualitative or quantitative reduction (e.g., by at least 10%, 20%, 30%, 40%, 50%, 60%, 70%, 80%, 90%, 95%, or more) in the tumor or metastases size or reduce or prevent metastatic growth. Preferably, when a soluble endoglin compound (e.g., a soluble endoglin protein, or an active fragment, derivative, or analog thereof) is used to treat a subject with a metastatic cancer, it is generally provided in a therapeutically effective amount sufficient to prevent metastasis or to reduce metastatic disease or metastatic lesions, to inhibit development of new metastatic lesions after treatment has started, to increase the disease-free survival time between the disappearance of a tumor, or a metastases, and its reappearance, to prevent an initial or subsequent occurrence of a tumor or metastases, or to reduce any adverse symptom associated with a tumor or a metastases. In one preferred embodiment, the percent of cancerous or metastatic cells surviving the treatment is at least 10, 20, 40, 60, 80, or 100% lower than the initial number of cancerous or metastatic cells, as measured using any standard assay. Preferably, the decrease in the number of cancerous or metastatic cells induced by administration of a therapy of the invention is at least 2, 5, 10, 20, or 50-fold greater than the decrease in the number of non-cancerous or non-metastatic cells. In yet another preferred embodiment, the number of cancerous or metastatic cells present after administration of a therapy is at least 2, 5, 10, 20, or 50-fold lower than the number of cancerous or metastatic cells present after administration of a vehicle control. Preferably, the methods of the present invention result in a decrease of at least 10, 20, 40, 60, 80, or 100% in the size of a primary or metastatic tumor as determined using standard methods. Preferably, the cancer does not reappear or reappears after at least 2, 5, 10, 15, or 20 years. In another preferred embodiment, the length of time a patient survives after being diagnosed with cancer and treated with a therapy of the invention is at least 20, 40, 60, 80, 100, 200, or even 500% greater than (i) the average amount of time an untreated patient survives or (ii) the average amount of time a patient treated with another therapy survives. For the treatment of cancers associated with angiogenesis, an effective amount can be the amount sufficient to achieve any one or more of the following: a reduction or inhibition in the formation of new blood vessels and/or modulating the volume, diameter, length, permeability, or number of existing blood vessels. Preferably, the methods of the present invention result in a reduction or inhibition of at least 20, 40, 60, 80, or even 100% in the volume, diameter, length, permeability, and/or number of blood vessels as determined using standard methods.

By “tumor” or “cancer” is meant both benign and malignant growths of cancer. Preferably, the cancer is malignant. The cancer can be a non-solid tumor (e.g., a tumor that grows within the blood stream) or a solid tumor, which refers to one that grows in an anatomical site outside the bloodstream (in contrast, for example, to blood-borne tumors, such as lymphomas and leukemia) and requires the formation of small blood vessels and capillaries to supply nutrients, etc., to the growing tumor mass. Examples of solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organ, bladder, skin, sarcomas, brain tumors, and bone tumors.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

Other features and advantages of the invention will be apparent from the following Detailed Description, the drawings, and the claims.

### BRIEF DESCRIPTION OF THE DRAWINGS

FIG. 1 is a schematic showing the endoglin protein. SP: signal peptide; ZP: zona pellucida domain; CL: potential cleavage site (amino acid 437) for the release of soluble endoglin; TM: transmembrane domain; Cyto: cytoplasmic domain.

FIG. 2 is a Western blot showing endoglin protein levels in placental samples from two pre-eclamptic patients, p32 and p36, that presented to the Beth Israel Deaconess Medical Center in 2003 and maternal serum from a pregnant woman. The Western blot was probed using a N-terminal antibody obtained from Santa Cruz Biotechnology, Inc. (Santa Cruz, Calif.), that shows both the 110 kD band in the placenta and a smaller 63 kD band that is present in the placenta and the serum samples.

FIG. 3A shows the predicted cDNA sequence (SEQ ID NO: 1) of soluble endoglin. FIG. 3B shows the predicted amino acid sequence (SEQ ID NO: 2) of soluble endoglin. It should be noted that the sequence includes the leader peptide sequence that would normally be cleaved in the endoplasmic reticulum.

FIG. 4A is a western blot showing soluble endoglin after purification from the serum of pre-eclamptic patients. Fractions 4 and 5 eluted from the 44G4-IgG (anti-Eng) Sepharose were run on SDS-PAGE under reducing conditions and tested by western blot using a polyclonal antibody to endoglin. The eluted fractions were subjected to mass spectrometry analysis (3 runs). FIG. 4B shows the sequence of human endoglin (SEQ ID NO: 3). Peptides identified by mass spectrometry are shown in bold and underlined. The underlined amino acids represent the transmembrane domain of human cell surface endoglin. Note that the amino acid sequence numbering starts at 26 as amino acids 1-25 represents the leader peptide. All references to amino acids sequences of SEQ ID NO: 3 are numbered based on the numbering in this figure.

FIG. 5 is a representative autoradiogram and graph of a dose-dependent increase in [I¹²⁵] TGF-β 1 binding to Tβ RII on mouse endothelial cells. Treatment with 5 nM recombinant soluble endoglin significantly reduced binding at 50 pM and 100 pM (*P<0.05 vs. untreated group). Competition with 40×excess cold TGF-β1 in cells treated with 100 pM [I¹²⁵] TGF-β1 abolished receptor binding and served as background control.

FIG. 6 is a graph showing significantly increased TGF-β-induced activation of the Smad 2/3-dependent CAGA-Luc reporter construct transfected in HUVECs and inhibition by treatment with sEng. (n=3, **P<0.01 vs. sEng untreated group).

FIG. 7 is a graph showing the percent change in rat renal microvessel diameter in microvascular reactivity experiments in the presence of TGF-β1 (B1) and TGF-β3 (B3) from doses ranging from 200 pg/ml-200 ng/ml. These same experiments were repeated in the presence of soluble endoglin (E) at 1 μg/ml. These data presented are a mean of 4 independent experiments.

FIG. 8 is a series of graphs showing inhibition of TGF-β1-mediated vascular reactivity in mesenteric vessels by soluble endoglin. Microvascular reactivity of rat mesenteric microvessels was measured in the presence of TGF-β1 or TGF-β3 from 200 pg/ml to 200 ng/ml. The experiments were repeated in the presence of recombinant soluble endoglin at 1 μg/ml. The mean±standard error of 4 independent experiments is shown (upper panel). Also shown is the blocking effect of L-N^{G}-nitroarginine methyl ester (L-NAME) on TGF-β1 1 at 1 ng/ml (lower panel).

FIG. 9 is a graph showing the percent change in the vascular diameter of renal microvessels in the presence of 1 ng/ml of VEGF (V), TGF-β1 (B1), and the combination (V+B1). Also shown is the effect of this combination in the presence of 1 μg/ml each of sFlt1 (S) and soluble endoglin (E) (V+B1+S+E). The data represents a mean of 4 independent experiments.

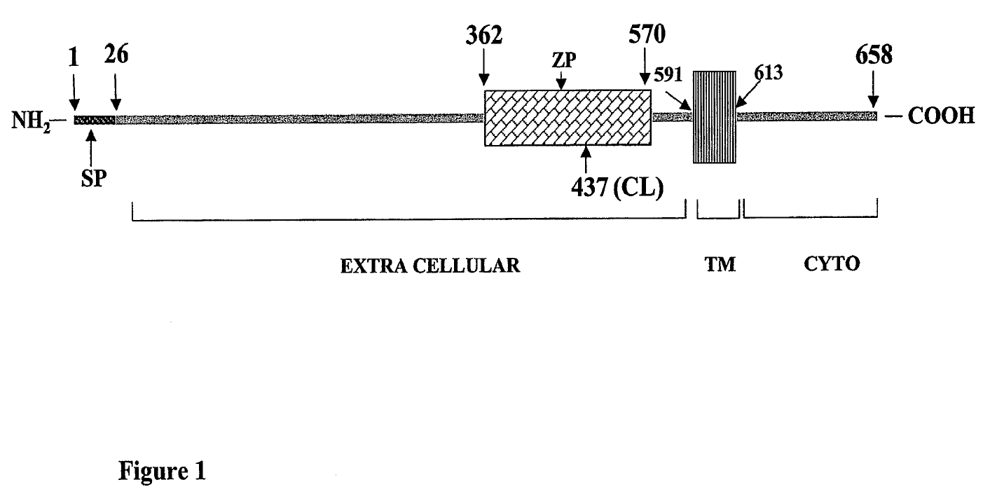

### DETAILED DESCRIPTION

The present invention is based on our discovery of a novel soluble form of endoglin, likely of placental origin, that is present in the sera of pregnant women. We have discovered that soluble endoglin interferes with TGF-β1 and TGF-β3 binding to its receptor leading to decreased signaling, such as a reduction in Smad2/3-dependent transcription using a reporter gene assay. We have also discovered that soluble endoglin compounds (e.g., soluble endoglin nucleic acid molecules, soluble endoglin proteins, or biologically active fragments, derivatives, or analogs thereof) can be used to treat or prevent cancers, such as those that overexpress TGF-β, or are associated with angiogenic activity.

TGF-β is the prototype of a family of at least 25 growth factors which regulate growth, differentiation, motility, tissue remodeling, neurogenesis, wound repair, apoptosis, and angiogenesis in many cell types. TGF-β also inhibits cell proliferation in many cell types and can stimulate the synthesis of matrix proteins. Soluble endoglin binds several specific members of the TGF-β family including TGF-β1, TGF-β3, activin, BMP-2, and BMP-7, and may serve to deplete the tumor cells or the surrounding environment of excess or dysfunctional TGF-β.

The present invention features methods of increasing the levels or biological activity of soluble endoglin and to neutralize the effects of TGF-β. The methods of the invention are useful for the treatment of any disease associated with increased TGF-β levels or aberrant TGF-β signaling. The methods are particularly useful for the treatment of cancer or for the treatment or prevention of cancer or cancer metastasis, for example by reversing the TGF-β pro-angiogenic activity. Non-limiting examples of such diseases and such TGF-β activity are described in U.S. Patent Application Publication Nos. 20050276802, 20050267021, and 20040131616, herein incorporated by reference.

Soluble endoglin compounds useful in the methods of the invention include any soluble endoglin polypeptide, or biologically active homologs, fragments, derivatives, or analogs thereof, or a nucleic acid sequence encoding a soluble endoglin polypeptide, or homolog, fragment, derivative, or analog thereof, wherein the polypeptide has an amino acid sequence that is substantially identical to at least a part of soluble endoglin (e.g., SEQ ID NO: 2, amino acids 1 to 587 of SEQ ID NO: 3, and amino acids 40 to 406 of SEQ ID NO: 3 for amino acid sequences, and SEQ ID NO: 1 for nucleic acid sequences) and has soluble endoglin biological activity (see below). Modifications to the primary structure itself by deletion, addition, or alteration of the amino acids incorporated into the soluble endoglin sequence during translation can be made without destroying the activity of the protein. Such modifications can be made to improve expression, stability, solubility, cellular uptake, or biological activity of the protein in the various expression systems. For example, a mutation can increase the binding of soluble endoglin to TGF-β or TGF-β receptor. Generally, substitutions are made conservatively and take into consideration the effect on biological activity. Mutations, deletions, or additions in nucleotide sequences constructed for expression of derivative or analog proteins or fragments thereof must, of course, preserve the reading frame of the coding sequences and preferably will not create complementary regions that could hybridize to produce secondary mRNA structures such as loops or hairpins which would adversely affect translation of the mRNA.

Additional useful soluble endoglin compounds include any peptidyl or non-peptidyl compound that is a soluble endoglin fragment, derivative, or analog and has or induces soluble endoglin biological activity; any compound known to stimulate or increase blood serum levels of soluble endoglin polypeptides or increase the biological activity of soluble endoglin polypeptides; any compound known to increase the expression of an enzyme, such as a metalloproteinase, responsible for the conversion of endoglin to soluble endoglin; any compound known to decrease the expression or biological activity of a inhibitor of soluble endoglin, such as an MMP inhibitor; and any soluble endoglin mimetic compound.

Compounds useful in the methods of the invention will increase soluble endoglin expression levels or biological activity by at least 10%, 20%, 30%, 40%, 50%, 60%, 70%, 80%, 90%, or more. Soluble endoglin expression levels can be determined using the assays described below. Soluble endoglin biological activity includes binding to a TGF-β family member (e.g., TGF-β1, TGF-β3, activin, and BMP), binding to a TGF-β receptor (e.g., TβRI and TβRII), blocking binding of TGF-β1 to TβRII, and inhibition of TGF-β signaling pathways. Assays for soluble endoglin biological activity include soluble endoglin-TGF-β binding assays or soluble endoglin-TβRII binding assays, TGF-β1 to TβRII binding assays, reporter gene assays for downstream signaling proteins such as Smad2/3. Binding assays are also well known in the art. For example, a BIAcore instrument can be used to determine the binding constant of a complex between two proteins. The dissociation constant for the complex can be determined by monitoring changes in the refractive index with respect to time as buffer is passed over the chip (O'Shannessy et al., Anal. Biochem. 212:457-468, 1993; Schuster et al:, Nature 365:343-347, 1993). Other suitable assays for measuring the binding of one protein to another include, for example, immunoassays such as enzyme-linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR). Additional examples of such assays are known in the art.

Soluble endoglin polypeptides can be produced by any of a variety of methods for protein production known in the art such as purification of naturally occurring soluble endoglin (e.g., from the placenta), products of chemical synthetic procedures, and products produced by recombinant techniques from a prokaryotic or eukaryotic host, including, for example, bacterial, fungus, higher plant, insect, and mammalian cells. In one example, soluble endoglin is produced by recombinant DNA methods by inserting a DNA sequence encoding soluble endoglin, or fragments, derivatives, or analogs thereof, into a recombinant expression vector and expressing the DNA sequence under conditions promoting expression. General techniques for nucleic acid manipulation are described, for example, by Sambrook et al., in “Molecular Cloning: A Laboratory Manual,” 2nd Edition, Cold Spring Harbor Laboratory press, 1989; Goeddel et al., in “Gene Expression Technology: Methods in Enzymology,” Academic Press, San Diego, Calif., 1990; Ausubel et al., in “Current Protocols in Molecular Biology,” John Wiley & Sons, New York, N.Y., 1998; Watson et al., “Recombinant DNA,” Chapter 12, 2nd edition, Scientific American Books, 1992; and other laboratory textbooks. The DNA encoding soluble endoglin is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

Appropriate cloning and expression vectors for use with bacterial, fungal, yeast, and mammalian cellular hosts can be found, for example, in “Cloning Vectors: A Laboratory Manual,” Elsevier, New York, 1985, the relevant disclosure of which is hereby incorporated by reference.

The expression construct is introduced into the host cell using a method appropriate to the host cell, as will be apparent to one of skill in the art. The expression construct can be introduced for transient expression of the protein or stable expression by selecting cells using a selectable marker in order to generate a stable cell line that expresses the protein continuously. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells for expression of soluble endoglin from recombinant vectors include prokaryotes, fungal, mammalian cells, or insect cells.

In addition, soluble endoglin may be expressed in transgenic mammals (e.g., mice and bovines) such that the soluble endoglin is released into the milk of the transgenic animals. For example, the pBC1 Milk Expression Vector Kit (Genzyme Transgenics Corporation and Invitrogen Corporation) provides a milk expression vector which features a β-casein expression promoter upstream of restriction site sequences (for insertion of the expressed transgene). Methods of making transgenic animals and the purification of transgenically expressed soluble proteins from the milk of such animals are known in the art.

Purified soluble endoglin, or biologically active fragments, derivatives, or analogs thereof, are prepared by culturing suitable host/vector systems to express the recombinant proteins. As a secreted protein, soluble endoglin is likely to be released from the membrane and can then be purified from culture media or cell extracts. If desired, a matrix metalloproteinase (MMP) may be added to enhance cleavage and release of soluble endoglin, examples of which are described below.

In one example, supernatants from systems which secrete recombinant protein into culture media can be first concentrated using a commercially available protein concentration filter, for example, an Amicon or Millipore Pellicon ultrafiltration unit, and then purified.

In addition to the methods employing recombinant DNA, soluble endoglin polypeptides, or fragments, derivatives, or analogs thereof, can be purified from sources that naturally produce the soluble form of the protein. Examples of these sources include any mammalian tissue or cells, such as placental tissues. The soluble endoglin from these sources can be purified and concentrated using any of the methods known in the art or described above.

After purification, soluble endoglin may be exchanged into different buffers and/or concentrated by any of a variety of methods known in the art, including, but not limited to, filtration and dialysis. The purified soluble endoglin is preferably at least 80% or 85% pure, more preferably at least 90% or 95% pure, and most preferably at least 98% pure. Regardless of the exact numerical value of the purity, the soluble endoglin is sufficiently pure for use as a pharmaceutical product.

Soluble endoglin polypeptides, or fragments, derivatives, or analogs thereof, can also be produced by chemical synthesis (e.g., by the methods described in “Solid Phase Peptide Synthesis,” 2nd ed., The Pierce Chemical Co., Rockford, Ill., 1984). Modifications to the protein, such as those described below, can also be produced by chemical synthesis.

Soluble Endoglin Modifications

The invention encompasses soluble endoglin polypeptides, or biologically active fragments, derivatives, or analogs thereof, which are modified during or after synthesis or translation. Modifications may provide additional advantages such as increased affinity, decreased off-rate, increased solubility, stability, and in vivo or in vitro circulating time of the polypeptide, or decreased immunogenicity and include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, for example, Creighton, “Proteins: Structures and Molecular Properties,” 2nd Ed., W. H. Freeman and Co., N.Y., 1992; “Postranslational Covalent Modification of Proteins,” Johnson, ed., Academic Press, New York, 1983; Seifter et al.; Meth. Enzymol., 182:626-646, 1990; Rattan et al., Ann. NY Acad. Sci., 663:48-62, 1992). Additionally, the soluble endoglin polypeptide may contain one or more non-classical amino acids. Non-classical amino acids include, but are not limited to, to the D-isomers of the common amino acids, 2,4-diaminobutyric acid, α-amino-isobutyric acid, 4-aminobutyric acid, Abu, 2-amino-butyric acid, g-Abu, e-Ahx, 6-amino-hexanoic acid, Aib, 2-amino-isobutyric acid, 3-amino-propionic acid, omithine, norleucine, norvaline, hydroxyproline, sarcosine, citrulline, homocitrulline, cysteic acid, t-butylglycine, t-butylalanine, phenylglycine, cyclohexylalanine, α-alanine, fluoro-amino acids, designer amino acids such as α-methyl amino acids, Ca-methyl amino acids, Na-methyl amino acids, and amino acid analogs in general. Furthermore, the amino acid can be D (dextrorotary) or L (levorotary).

Additional post-translational modifications encompassed by the invention include, for example, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression.

As described above, the invention also includes chemically modified derivatives of soluble endoglin, which may provide additional advantages such as increased solubility, stability, and circulating time of the polypeptide, or decreased immunogenicity (see U.S. Pat. No. 4,179,337). The chemical moieties for derivitization may be selected from water soluble polymers such as, for example, polyethylene glycol, ethylene glycol/propylene glycol copolymers, carboxymethylcellulose, dextran, polyvinyl alcohol and the like. The soluble endoglin polypeptide may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). As noted above, the polyethylene glycol may have a branched structure. Branched polyethylene glycols are described, for example, in U.S. Pat. No. 5,643,575; Morpurgo et al., Appl. Biochem. Biotechnol. 56:59-72, 1996; Vorobjev et al., Nucleosides Nucleotides 18:2745-2750, 1999; and Caliceti et al., Bioconjug. Chem. 10:638-646, 1999, the disclosures of each of which are incorporated by reference.

The polyethylene glycol molecules (or other chemical moieties) should be attached to the soluble endoglin polypeptide with consideration of effects on functional or antigenic domains of the protein. There are a number of attachment methods available to those skilled in the art, e.g., EP 0401384 (coupling PEG to G-CSF), herein incorporated by reference, see also Malik et al., Exp. Hematol. 20:1028-1035, 1992 (reporting pegylation of GM-CSF using tresyl chloride). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues, and the C-terminal amino acid residue. Sulfhydryl groups may also be used as a reactive group for attaching the polyethylene glycol molecules. Preferred for therapeutic purposes is attachment at an amino group, such as attachment at the N-terminus or a lysine residue. The number of polyethylene glycol moieties attached to each polypeptide of the invention (i.e., the degree of substitution) may also vary. For example, the pegylated soluble endoglin may be linked, on average, to 1, 2, 3, 4, 5, 6, 7, 8, 9, 10, 12, 15, 17, 20 or more polyethylene glycol molecules. Similarly, the average degree of substitution may range within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per polypeptide molecule. Methods for determining the degree of substitution are discussed, for example, in Delgado et al., Crit. Rev. Thera. Drug Carrier Sys., 9:249-304, 1992.

The soluble endoglin polypeptides may also be modified with a detectable label, including, but not limited to, an enzyme, prosthetic group, fluorescent material, luminescent material, bioluminescent material, radioactive material, positron emitting metal, nonradioactive paramagnetic metal ion, and affinity label for detection and isolation of a soluble endoglin target. The detectable substance may be coupled or conjugated either directly to the polypeptides of the invention or indirectly, through an intermediate (such as, for example, a linker known in the art) using techniques known in the art. Examples of suitable enzymes include horseradish peroxidase, alkaline phosphatase, β-galactosidase, glucose oxidase, or acetylcholinesterase; examples of suitable prosthetic group complexes include streptavidin/biotin and avidin/biotin; examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I), carbon (¹⁴C), sulfur (³⁵S), tritium (³H), indium (¹¹⁵mIn, ¹¹³mIn, ¹¹²In, ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, Lu, ¹⁵⁹Gd, ¹⁴⁹Pm, ⁴⁰La, ¹⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, and tin (¹¹³Sn, ¹¹⁷Sn). The detectable substance may be coupled or conjugated either directly to the soluble endoglin polypeptide or indirectly, through an intermediate (such as, for example, a linker known in the art) using techniques known in the art. See, for example, U.S. Pat. No. 4,741,900 for metal ions, which can be conjugated to soluble endoglin polypeptide for use as diagnostics according to the present invention.

The soluble endoglin polypeptide can also be modified by conjugation to another protein or therapeutic compound. Such conjugation can be used, for example, to enhance the stability or solubility of the protein, to reduce the antigenicity, or to enhance the therapeutic effects of the protein. A preferred fusion protein comprises a heterologous region from immunoglobulin (e.g., all or part of the Fc region) that is useful to solubilize proteins (see, EP-A 0232 262).

A soluble endoglin polypeptide of the invention may be conjugated to a therapeutic moiety such as a cytotoxin, e.g., a cytostatic or cytocidal agent, a chemotherapeutic agent, a radiotherapeutic agent, or a radioactive metal ion, e.g., alpha-emitters such as, for example, ²¹³Bi, or other radioisotopes such as, for example, ¹⁰³Pd, ¹³³Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ⁹⁰Yttrium, ¹¹⁷Tin, ¹⁸⁶Rhenium, ¹⁶⁶Holmium, and ¹⁸⁸Rhenium.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and fragments, variants or homologs thereof.

Additional therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU), lomustine (CCNU), cyclophosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label soluble endoglin polypeptides of the invention. Such techniques include, but are not limited to, the use of bifunctional conjugating agents (see, e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the relevant disclosures of each of which are hereby incorporated by reference in its entirety) and direct coupling reactions (e.g., Bolton-Hunter and Chloramine-T reaction).

The invention also includes mimetics, based on modeling the 3-dimensional structure of a polypeptide or peptide fragment and using rational drug design to provide potential inhibitor compounds with particular molecular shape, size, and charge characteristics. Following identification of a therapeutic compound, suitable modeling techniques known in the art can be used to study the functional interactions and design mimetic compounds which contain functional groups arranged in such a manner that they could reproduce those interactions. The designing of mimetics to a known pharmaceutically active compound is a known approach to the development of pharmaceuticals based on a lead compound. This might be desirable where the active compound is difficult or expensive to synthesize or where it is unsuitable for a particular method of administration, e.g., peptides are not well suited as active agents for oral compositions as they tend to be quickly degraded by proteases in the alimentary canal. Mimetic design, synthesis, and testing may be used to avoid randomly screening a large number of molecules for a target property. The mimetic or mimetics can then be screened to see whether they increase soluble endoglin levels or biological activity, and further optimization or modification can then be carried out to arrive at one or more final mimetics for in vivo or clinical testing.

Therapeutic Compounds that Simulate Soluble Endoglin Production

We have identified a potential cleavage site in the extracellular domain of endoglin where a proteolytic enzyme could cleave the membrane-bound form of endoglin, releasing the extracellular domain as a soluble form. Our sequence alignments of the cleavage site suggest that a matrix metalloproteinase (MMP) may be responsible for the cleavage and release of soluble endoglin. Alternatively, a cathepsin or an elastase may also be involved in the cleavage event. MMPs are also known as collagenases, gelatinases, and stromelysins, and there are currently 26 family members known (for a review see, Whittaker and Ayscough, Cell Transmissions 17:1, 2001). A preferred MMP is MMP9, which is known to be upregulated in placentas from pre-eclamptic patients (Lim et al., Am. J. Pathol. 151:1809-1818, 1997). Candidate MMPs can be tested in vitro to determine if they can cleave endoglin, specifically membrane-bound endoglin, to produce soluble endoglin. For these assays, a candidate MMP can be added to a culture of cells expressing endoglin and the presence of soluble endoglin after addition of the candidate MMP can be assayed using standard techniques known in the art, for example Western blotting using an antibody specific for soluble endoglin. MMPs that enhance or upregulate soluble endoglin levels can then be used in the therapeutic methods of the invention, for example, as a purified protein (e.g., recombinant, synthetic, or purified from a natural source), or biologically active fragment thereof, or as a nucleic acid encoding the MMP.

Therapeutic Compounds that Decrease MMP Inhibitor Expression or Biological Activity

The activity of MMPs is controlled through activation of pro-enzymes and inhibition by endogenous inhibitors such as the tissue inhibitors of metalloproteinases (TIMPS). Inhibitors of MMPs are zinc-binding proteins. There are four known endogenous inhibitors (TIMP 1-4), which are reviewed in Whittaker et al., supra. One preferred MMP inhibitor is the inhibitor of membrane type-MMP1 that has been shown to cleave betaglycan, a molecule that shares similarity to endoglin (Velasco-Loyden et al., J. Biol. Chem. 279:7721-7733, 2004). Given the potential role of MMPs, cathepsins, or elastases in the release and up-regulation of soluble endoglin levels, the present invention also provides for the use of any compound, that can decrease the expression or biological activity of any compound (e.g., protein), such as those described above, known to inhibit the activity of any MMP, cathepsin, or elastase involved in the cleavage and release of soluble endoglin, for the treatment or prevention of cancer or cancer metastasis in a subject.

### Therapeutic Nucleic Acids

Nucleic acids encoding soluble endoglin, or fragments, derivatives, or analogs thereof, or MMPs, or inhibitors of MMP inhibitors can also be used in the therapeutic methods of the invention. The nucleic acids encoding the desired protein may be obtained using routine procedures in the art, e.g. recombinant DNA and PCR amplification. For any of the nucleic acid applications described herein, standard methods for administering nucleic acids can be used. Examples are described in U.S. Patent Application Publication No. 20060067937 and PCT Publication No. WO 06/034507.

### Therapeutic Applications

We have discovered that soluble endoglin inhibits the binding of TGF-β to its receptor and reduces or inhibits TGF-β signaling events. Soluble endoglin compounds (e.g., soluble endoglin nucleic acid molecules, soluble endoglin proteins, or biologically active fragments, derivatives, or analogs thereof) can be used to treat or prevent angiogenic or proliferative disorders that are characterized by increased TGF-β activity or expression levels such as cancer, particularly those cancers that are associated with angiogenic activity or both increased angiogenic activity and increased TGF-β activity or expression levels. In specific embodiments, a soluble endoglin compound can be used to treat proliferative diseases, such as cancer, where the angiogenic activity is TGF-β mediated. Examples of additional angiogenic disorders that can be treated or prevented by the therapeutic methods of the invention, include but not limited to, inflammatory disorders such as immune and non-immune inflammation, rheumatoid arthritis, ocular neovascular disease, choroidal retinal neovascularization, osteoarthritis, chronic articular rheumatism, psoriasis, disorders associated with inappropriate or inopportune invasion of vessels such as diabetic retinopathy, neovascular glaucoma, restenosis, capillary proliferation in atherosclerotic plaques and osteoporosis, cancer-associated disorders, and those described in U.S. Patent Application Publication No. 20040131616, herein incorporated by reference, and additional angiogenic disorders described herein.

TGF-β has been shown to be upregulated in several types of cancers (e.g., breast, colorectal, renal, prostate, and endometrial) and is thought to contribute to the angiogenesis associated with tumor development and metastasis. Accordingly, the invention includes the use of soluble endoglin compounds (e.g., a soluble endoglin proteins, or biologically active fragments, derivatives, or analogs thereof) to treat, prevent, or reduce cancer and, particularly, cancer metastasis. Of particular importance to the present invention are subjects (e.g., humans and other mammals) diagnosed with and/or treated for a primary tumor, including prophylactic treatment of at-risk subjects, not yet diagnosed with metastatic disease or determined to lack metastatic disease, and those subjects otherwise predisposed to developing metastatic disease. The methods of the invention can be used to prevent the occurrence or re-occurrence of metastatic disease. Also included are subjects who have undergone treatment for metastasis or a possible metastasis in order to prevent or reduce metastatic disease. The methods of the invention can be used before, during, or after additional therapies to treat the primary tumor, the metastases, or the risk of either.

The term cancer embraces a collection of malignancies with each cancer of each organ consisting of numerous subsets. Typically, at the time of cancer diagnosis, “the cancer” consists in fact of multiple subpopulations of cells with diverse genetic, biochemical, immunologic, and biologic characteristics. Benign or malignant growths of cancer are referred to as tumors. The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, mature B-cell acute lymphoblastic leukemia, chronic lymphocytic leukemia, prolymphocytic leukemia, or hairy cell leukemia), or lymphoma (e.g., non-Hodgkin's lymphoma, cutaneous T-cell lymphoma, or Hodgkin's disease). Solid tumors can be further separated into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, endometrium, male genital organ, urinary organs, bladder, and skin. Solid tumors of non-epithelial origin include sarcomas, brain tumors, and bone tumors. While the methods of the invention can be used to treat any tumor or tumor metastasis, a soluble endoglin compound (e.g., a soluble endoglin protein, or a biologically active fragment, derivative, or analog thereof) is preferably used for the treatment or prevention of cancers that have increased TGF-β levels or biological activity, particularly TGF-β1 or TGF-β3, and/or tumors that have angiogenic activity.

### Combination Therapies

In various embodiments soluble endoglin nucleic acids or polypeptides can be provided in conjunction (e.g., before, during, or after) with additional cancer therapies to prevent or reduce tumor growth or metastasis. Treatment therapies include but are not limited to surgery, radiation therapy, chemotherapy, immune therapy (e.g., cytokines, cancer-specific antibodies, interferons, and biologics), differentiating therapy, anti-angiogenic therapy, hormone therapy, or hyperthermia. Soluble endoglin compounds (e.g., a soluble endoglin proteins, or biologically active fragments, derivatives, or analogs thereof) may be formulated alone or in combination with any additional cancer therapies in a variety of ways that are known in the art. Such additional cancer therapies can be administered before, during, or after the administration of the soluble endoglin compounds of the invention.

In addition, the invention provides for the use of an angiogenesis inhibitor used in combination with any of the soluble endoglin compounds described herein to treat cancer or cancer metastasis. Angiogenesis inhibitors, also known as anti-angiogenic agents, that may be used in combination with any of the soluble endoglin compounds include: an anti-angiogenic antibody (e.g., an antibody that binds VEGF-A or an antibody that binds a VEGF receptor and blocks VEGF binding (e.g., avastin and those described in U.S. Patent Publication Nos. 20030175271, 20050186208, 20060030529, 20070025999, 20070036753,2007003654, 20070036755, 20070036790, 20070071718, 20070071748, and 20070071749)), VEGF trap, soluble VEGF receptor (e.g., sFlt1 and those described in U.S. Pat. Nos. 5,712,380; 5,861,484; and 7,071,159; and U.S. Patent Publication Nos. 20030120038, 20050276808, and 20070037748), endostatin, angiostatin, restin, tumstatin, TNP-470, 2-methoxyestradiol, thalidomide, antibodies that inhibit TGF-β biological activity, a peptide fragment of an anti-angiogenic protein, canstatin, arrestin, a VEGF kinase inhibitor (e.g., SU11248, PTK787, BAY 43-9006, 1,5-diarylbenzimidazoles, and the inhibitors disclosed in U.S. Pat. Nos. 6,448,277; 6,465,484; and 7,045,133; and U.S. Patent Publication Nos. 20050085637, 20050234083, 20050288515, 20060135501, 20060160861, 20060264425, and 20070015756), CPTK787, SFH-1, an anti-angiogenic protein, thrombospondin-1, platelet factor-4, interferon-α, an agent that blocks TIE-1 or TIE-2 signaling, an agent that blocks PIH12 signaling, an agent that blocks an extracellular vascular endothelial (VE) cadherin domain, an antibody that binds to an extracellular VE-cadherin domain, tetracycline, penicillamine, vinblastine, cytoxan, edelfosine, tegafur or uracil, curcumin, green tea, genistein, resveratrol, N-acetyl cysteine, captopril, a COX-2 inhibitor, celecoxib, and rofecoxib.

Given our data described below indicating cooperation between the TGF-β and VEGF signaling pathways, preferred combinations will include a soluble endoglin compound (e.g., a soluble endoglin protein, or a biologically active fragment, derivative, or analog thereof) in combination with a VEGF inhibitor or VEGF antagonist as described above (e.g., avastin, VEGF trap, sFlt1, or an antibody that specifically binds VEGF).

The dosage of the angiogenesis inhibitor will depend on other clinical factors such as weight and condition of the human or animal and the route of administration of the compound. For treating humans or animals, between approximately 0.5 mg/kg to 500 mg/kg body weight of the angiogenesis inhibitor can be administered. A more preferable range is 1 mg/kg to 100 mg/kg body weight with the most preferable range being from 2 mg/kg to 50 mg/kg body weight. Depending upon the half-life of the angiogenesis inhibitor in the particular animal or human, the angiogenesis inhibitor can be administered between several times per day to once a week. The methods of the present invention provide for single as well as multiple administrations, given either simultaneously or over an extended period of time.

In addition, the invention provides for the use of an anti-proliferative compound used in combination with any of the soluble endoglin compounds (e.g., soluble endoglin proteins, or biologically active fragments, derivatives, or analogs thereof) for treating a tumor. Anti-proliferative compounds that may be used in combination with any of the soluble endoglin compounds include taxol, troglitazone, an antibody that binds basic fibroblast growth factor (bFGF), an antibody that binds bFGF-saporin, a statin, an acetylcholinesterase (ACE) inhibitor, suramin, 17-beta-estradiol, atorvastatin, fluvastatin, lovastatin, pravastatin, simvastatin, cerivastatin, perindopril, quinapril, captopril, lisinopril, enalapril, fosinopril, cilazapril, ramipril, and a kinase inhibitor.

The dosage of the anti-proliferative compound depends on clinical factors such as weight and condition of the human or animal and the route of delivery of the compound. In general, for treating humans or animals, between approximately 0.1 mg/kg to 500 mg/kg body weight of the anti-proliferative compound can be administered. A more preferable range is 1 mg/kg to 50 mg/kg body weight with the most preferable range being from 1 mg/kg to 25 mg/kg body weight. Depending upon the half-life of the anti-proliferative compound in the particular animal or human, the compound can be administered between several times per day to once a week. The methods of the present invention provide for single as well as multiple administrations, given either simultaneously or over an extended period of time.

It should be noted that although each of the compounds is listed under a specific category of compounds, these categories are not meant to be limiting in scope. Many of the compounds possess more than one activity and can therefore be included under more than one category.

For each of the compounds listed, all of the modes of administration described herein can be used. As some of the compounds described have shown toxicity when administered orally or systemically, local administration can also be used. In general, percent composition of the compound will range from 0.05% to 50% weight for weight of compound to coating material used.

### Therapeutic Formulations

The soluble endoglin compounds of the present invention (e.g., soluble endoglin proteins, or biologically active fragments, derivatives, or analogs thereof) can be formulated and administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. The soluble endoglin compound can be in the form of a pill, tablet, capsule, liquid, or sustained-release tablet for oral administration; or a liquid for intravenous or subcutaneous, systemic administration; or a polymer or other sustained-release vehicle for local administration.

The soluble endoglin compounds can be administered continuously by infusion, using a constant- or programmable-flow implantable pump, or by periodic injections. Sustained-release systems can also be used. Administration can be continuous or periodic. Semi-permeable, implantable membrane devices are also useful as a means for delivering soluble endoglin in certain circumstances. For example, cells that secrete soluble endoglin can be encapsulated, and such devices can be implanted into a subject, for example, into a primary tumor (e.g., a head and neck cancer or a pancreatic or esophageal cancer). In another embodiment, the soluble endoglin compound is administered locally, e.g., by direct injections, when the disorder or location of the tumor permits, and the injections can be repeated periodically. Such local administration is particularly useful in the prevention and treatment of local metastasis.

Therapeutic formulations are prepared using standard methods known in the art by mixing the active ingredient having the desired degree of purity with optional physiologically acceptable carriers, excipients, or stabilizers (Remington's Pharmaceutical Sciences (20^{th} edition), ed., A. Gennaro, 2000, Lippincott, Williams & Wilkins, Philadelphia, Pa.), in the form of lyophilized formulations or aqueous solutions. Acceptable carriers, include saline, or buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

In one exemplary in vivo approach, the soluble endoglin compound is a soluble endoglin polypeptide. The soluble endoglin polypeptide can be delivered systemically to the subject or directly to the tumor cells, e.g., to a tumor or a tumor bed following surgical excision of the tumor, in order to prevent or reduce metastasis or to inhibit survival of any remaining tumor or metastases cells. The dosage required depends on the choice of the route of administration; the nature of the formulation; the nature of the subject's illness; the subject's size, weight, surface area, age, and sex; other drugs being administered; and the judgment of the attending physician.

Wide variations in the needed dosage are to be expected in view of the variety of polypeptides and fragments, derivatives, and analogs available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2, 3, 6, 8, 10, 20, 50, 100, 150, or more). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding a soluble endoglin polypeptide can be delivered to the appropriate cells in the subject. Expression of the coding sequence can be directed to any cell in the body of the subject. In certain embodiments, expression of the coding sequence can be directed to the tumor or metastases themselves. This can be achieved by, for example, the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2nd edition, Scientific American Books, 1992). Examples of methods of gene delivery include liposome-mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of soluble endoglin.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic forces or covalent bonds. Poly-L-lysine binds to a ligand that can bind to a receptor on target cells (Cristiano et al., J. Mol. Med. 73:479, 1995). Alternatively, tissue specific targeting can be achieved by the use of tissue-specific transcriptional regulatory elements which are known in the art. Delivery of “naked DNA” (i.e., without a delivery vehicle) to an intramuscular, intradermal, or subcutaneous site is another means to achieve in vivo expression.

Gene delivery using viral vectors such as adenoviral, retroviral, lentiviral, or adeno-asociated viral vectors can also be used. Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opin. Biotechnol. 1:55-61, 1990; Sharp, The Lancet 337:1277-1278, 1991; Cornetta et al., Nucleic Acid Res. Mol. Biol. 36:311-322,1987; Anderson, Science 226:401-409, 1984; Moen, Blood Cells 17:407-416, 1991; Miller and Rosman, Biotechniques 7:980-990, 1989; Rosenberg et al., N. Engl. J Med. 323:370, 1990; Groves et al., Nature, 362:453-457, 1993; Horrelou et al., Neuron 5:393-402, 1990; Jiao et al., Nature 362:450-453, 1993; Davidson et al., Nature Genetics 3:2219-2223, 1993; Rubinson et al., Nature Genetics 33, 401-406, 2003; and U.S. Pat. Nos. 6,180,613; 6,410,010; and 5,399,346; all hereby incorporated by reference). These vectors include adenoviral vectors and adeno-associated virus-derived vectors, retroviral vectors (e.g., Moloney Murine Leukemia virus based vectors, Spleen Necrosis Virus based vectors, Friend Murine Leukemia based vectors, lentivirus based vectors (Lois et al., Science, 295:868-872, 2002; Rubinson et al., supra), papova virus based vectors (e.g., SV40 viral vectors), and Herpes-Virus based vectors), viral vectors that contain or display the Vesicular Stomatitis Virus G-glycoprotein Spike, Semliki-Forest virus based vectors, Hepadnavirus based vectors, and Baculovirus based vectors.

In the relevant polynucleotides (e.g., expression vectors), the nucleic acid sequence encoding the soluble endoglin polypeptide (including an initiator methionine and optionally a targeting sequence) is operatively linked to a promoter or enhancer-promoter combination. Short amino acid sequences can act as signals to direct proteins to specific intracellular compartments. Such signal sequences are described in detail in U.S. Pat. No. 5,827,516, herein incorporated by reference.

An ex vivo strategy can also be used for therapeutic applications. Ex vivo strategies involve transfecting or transducing cells obtained from the subject with a polynucleotide encoding a soluble endoglin polypeptide (or a biologically active fragment, derivative, or analog thereof). The transfected or transduced cells are then returned to the subject. The cells can be any of a wide range of types including, without limitation, hemopoietic cells (e.g., bone marrow cells, macrophages, monocytes, dendritic cells, T-cells, or B-cells), fibroblasts, epithelial cells, endothelial cells, keratinocytes, or muscle cells. Such cells act as a source of the soluble endoglin polypeptide for as long as they survive in the subject. Alternatively, tumor cells (e.g., any of those listed herein), preferably obtained from the subject but potentially from an individual other than the subject, can be transfected or transformed by a vector encoding a soluble endoglin polypeptide. The tumor cells, preferably treated with an agent (e.g., ionizing irradiation) that ablates their proliferative capacity, are then introduced into the patient, where they secrete exogenous soluble endoglin.

The ex vivo methods include the steps of harvesting cells from a subject, culturing the cells, transducing them with an expression vector, and maintaining the cells under conditions suitable for expression of the soluble endoglin polypeptide (or a biologically active fragment, derivative, or analog thereof). These methods are known in the art of molecular biology. The transduction step is accomplished by any standard means used for ex vivo gene therapy including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

The dosage and the timing of administering the compound depends on various clinical factors including the overall health of the subject and the severity of the symptoms. In general, once a tumor, metastatic disease, or a propensity to develop a tumor or metastatic is detected, any of the methods for administering the compound described herein can be used to treat or prevent further progression of the condition. For example, continuous systemic infusion or periodic injection to the site of the tumor or metastasis of the soluble endoglin compound (e.g., a soluble endoglin protein, or a biologically active fragment, derivative, or analog thereof) can be used to treat or prevent the disorder. Treatment can be continued for a period of time ranging from 1 day through the lifetime of the subject, more preferably 1 day to 5 years, 1 day to 1 year, 1 to 100 days, and most preferably 1 to 20 days. For treating subjects, between approximately 0.1 mg/kg to 500 mg/kg body weight of the soluble endoglin compound can be administered. A more preferable range is 1 mg/kg to 50 mg/kg body weight with the most preferable range being from 1 mg/kg to 25 mg/kg body weight. Depending upon the half-life of the soluble endoglin compound in the particular subject, the compound can be administered between several times per day to once a week. The methods of the present invention provide for single as well as multiple administrations, given either simultaneously, or over an extended period of time.

Where sustained-release administration of a soluble endoglin compound is desired in a formulation with release characteristics suitable for the treatment of any disease or disorder requiring administration of the soluble endoglin compound, microencapsulation of the soluble endoglin compound is contemplated. Micro-encapsulation of recombinant proteins for sustained release has been successfully performed with human growth hormone (rhGH), interferon-γ (rhIFN-γ), interleukin-2, and MN rgp120 (see, for e.g., Johnson et al., Nat. Med., 2:795-799, 1996; Yasuda, Biomed. Ther., 27:1221-1223, 1993; Hora et al., Bio/Technology, 8:755-758 1990; Cleland, “Design and Production of Single Immunization Vaccines Using Polylactide Polyglycolide Microsphere Systems,” in “Vaccine Design: The Subunit and Adjuvant Approach,” Powell and Newman, eds., Plenum Press: New York, pp. 439-462, 1995; WO 97/03692; WO 96/40072; WO 96/07399; and U.S. Pat. No. 5,654,010; herein incorporated by reference).

The sustained-release formulations may include those developed using poly(lactic-co-glycolic acid) (PLGA) polymer. The degradation products of PLGA, lactic and glycolic acids, can be cleared quickly within the human body. Moreover, the degradability of this polymer can be adjusted from months to years depending on its molecular weight and composition. See, Lewis, “Controlled release of bioactive agents from lactide/glycolide polymer,” in M. Chasin and Dr. Langer (Eds.), Biodegradable Polymers as Drug Delivery Systems (Marcel Dekker: New York, pp. 1-41, 1990).

The soluble endoglin for use in the present invention may also be modified in a way to form a chimeric molecule comprising soluble endoglin fused to another, heterologous polypeptide or amino acid sequence, such as an Fc sequence (e.g., a soluble endoglin immunoadhesin), or an additional therapeutic molecule (e.g., a chemotherapeutic or cytotoxic agent).

The soluble endoglin compound can be packaged alone or in combination with other therapeutic compounds as a kit (e.g., a chemotherapeutic agent, an angiogenesis inhibitor, or an anti-proliferative compound). Non-limiting examples include kits that contain, e.g., two pills, a pill, and a powder, a suppository, a liquid in a vial, two topical creams, etc.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Identification of New Compounds or Extracts

In general, compounds capable of increasing the expression levels or biological activity of soluble endoglin are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their molt-disrupting activity should be employed whenever possible.

When a crude extract is found to increase the biological activity or expression levels of a soluble endoglin polypeptide, further fractionation of the positive lead extract is necessary to isolate chemical constituents responsible for the observed effect. Thus, the goal of the extraction, fractionation, and purification process is the careful characterization and identification of a chemical entity within the crude extract that increases the biological activity or expression levels of a soluble endoglin. Methods of fractionation and purification of such heterogeneous extracts are known in the art. If desired, compounds shown to be useful as therapeutics for the treatment of cancer, particular cancers associated with elevated TGF-β levels and/or angiogenesis, are chemically modified according to methods known in the art.

Any number of methods are available for carrying out screening assays to identify new candidate compounds that modulate the expression or biological activity of a soluble endoglin polypeptide or nucleic acid molecule. Examples are described in detail in U.S. Patent Application Publication No. 2006/0067937 and PCT Publication No. WO 06/034507, herein incorporated by reference.

In one working example, candidate compounds may be screened for those that specifically bind to a TGF-β (e.g., TGF-β1 or TGF-β3) or TGF-β receptor (e.g., TβRI and TβRII) polypeptide. The efficacy of such a candidate compound is dependent upon its ability to interact with such a polypeptide or a functional equivalent thereof. Such an interaction can be readily assayed using any number of standard binding techniques and functional assays such as immunoassays or affinity chromatography based assays, examples of which are described above. In one embodiment, a TGF-β1 polypeptide is immobilized and soluble endoglin compounds (e.g., mimetics, or fragments, derivatives, or analogs of soluble endoglin) are tested for the ability to bind to the immobilized TGF-β using standard affinity chromatography based assays. Compounds that bind to the immobilized TGF-β can then be eluted and purified and tested further for its ability to bind to TGF-β in vivo or in vitro or its ability to inhibit the biological activity of TGF-β or to increase the biological activity of soluble endoglin.

In another example, a candidate compound is tested for its ability to decrease the biological activity of a TGF-β polypeptide by decreasing binding of a TGF-β (e.g., TGF-β1 and TGF-β3) to a TGF-β receptor (e.g., TβRI and TβRII). These assays can be performed in vivo or in vitro and the biological activity of the TGF-β polypeptide can be assayed using any of the assays for any of the TGF-β activities known in the art or described herein. For example, cells can be incubated with a Smad2/3-dependent reporter construct. The cells can then be incubated in the presence of TGF-β which will activate the Smad2/3-dependent reporter construct. Candidate compounds can be added to the cell and any compound that results in a decrease of TGF-β-induced activation of the Smad2/3-dependent reporter in the soluble endoglin-treated cells as compared to cells not treated with the compound, is considered a compound that may be useful for the treatment of cancer or metastatic disease.

In another example, candidate compounds can be assayed for the ability to reverse TGF-β induced angiogenesis or to convert cells overexpressing TGF-β from a pro-angiogenic to an anti-angiogenic state. Examples of angiogenesis assays are known in the art and some are described below.

### EXAMPLES

The following examples are intended to illustrate the invention. They are not meant to limit the invention in any way.

### Example 1

### Characterization of Soluble Endoglin

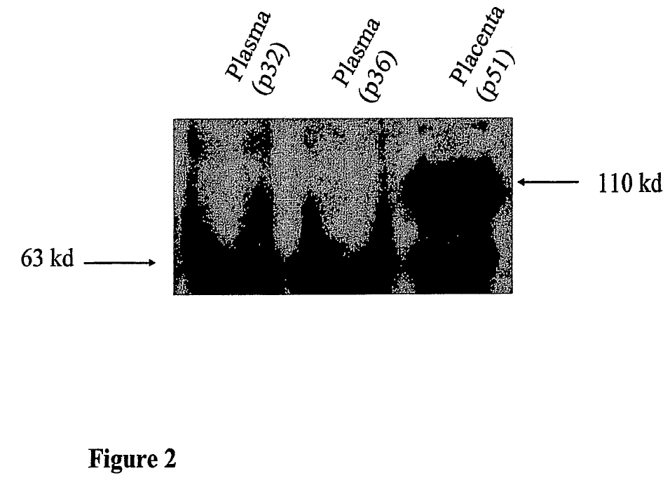

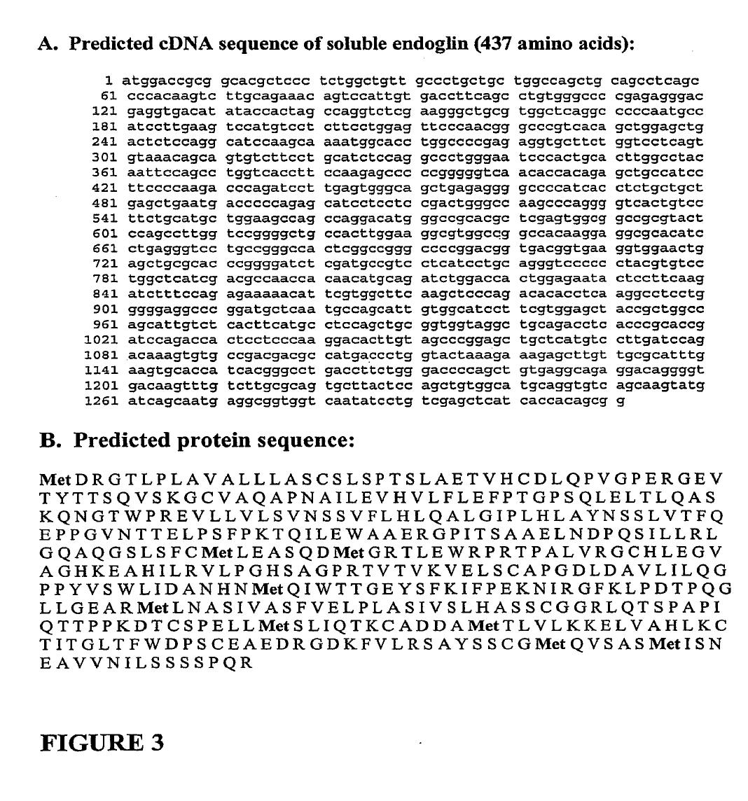

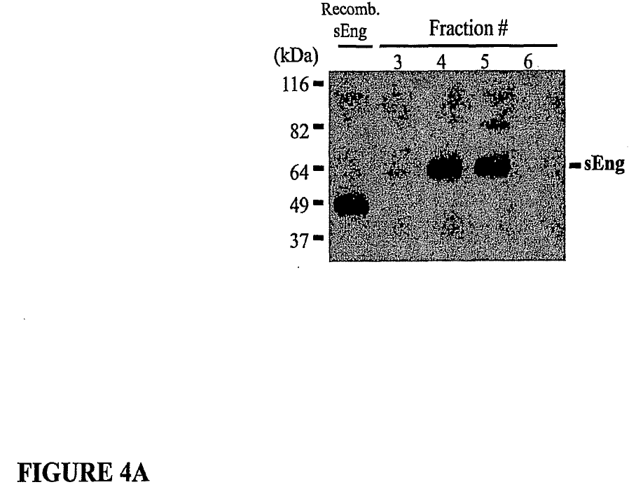

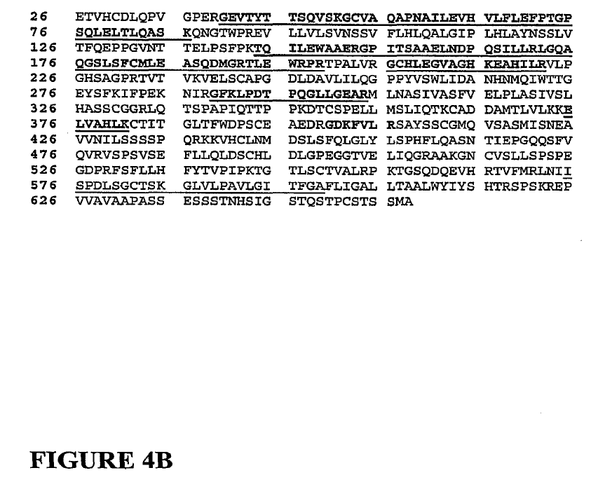

Placentas and serum from pre-eclamptic women were analyzed by Western blot using anti-endoglin antibodies. In these experiments, we detected a smaller protein, approximately 63 kDa in size, that was present in the placenta and serum of pre-eclamptic pregnant women (FIG. 2 and FIG. 4A). This protein was present at much lower levels in the sera of normal pregnant women and barely detectable in non-pregnant women. We have demonstrated that this smaller fragment is the extracellular domain of endoglin. The predicted cDNA and amino acid sequence of soluble endoglin are shown in FIGS. 3A and 3B, respectively. This soluble form of endoglin may be acting as an anti-angiogenic agent by binding to circulating ligands that are necessary for normal vascular health.

We purified the soluble endoglin protein from sera of pre-eclamptic patients and analyzed it by mass spectrometry. Serum (10 ml) from pre-eclamptic patients was sequentially applied onto CM Affi-gel blue and protein A Sepharose (Bio-Rad) columns to remove albumin and immunoglobulins, respectively. The flow through was slowly applied to a 2.5 ml column of mAb 44G4 IgG to human Eng, conjugated to Sepharose. Bound fractions were eluted with 0.02 M diethylamine, pH 11.4 and immediately neutralized with 1 M Tris, pH 7.8. Fractions 4 and 5 with elevated absorbance at 280 nm were pooled, reduced with 10 mM DTT for 1 hour at 57° C., and alkylated with 0.055 M iodoacetomide. The samples were then completely digested with trypsin (1:100). The lyophilized sample was resuspended in 0.1% tri-fluoroacetic acid and injected in a CapLC (Waters) HPLC instrument. Peptides were separated using a 75 μm Nano Series column (LC Packings) and analyzed using a Qstar XL MS/MS system. The data was searched using the Mascot search engine (Matrix Science) against the human protein database, NCBInr. This analysis revealed several Eng-specific peptides ranging from Gly40 to Arg406 (FIG. 4B) indicating a soluble form (sEng) corresponding to the N-terminal region of the full-length protein. This truncated version is likely to be shed from the placental syncitiotrophoblasts and endothelial cells and circulated in excess quantities in patients with pre-eclampsia.

### Example 2

### Soluble Endoglin Inhibits TGF-β1 Binding and Signaling in Endothelial Cells

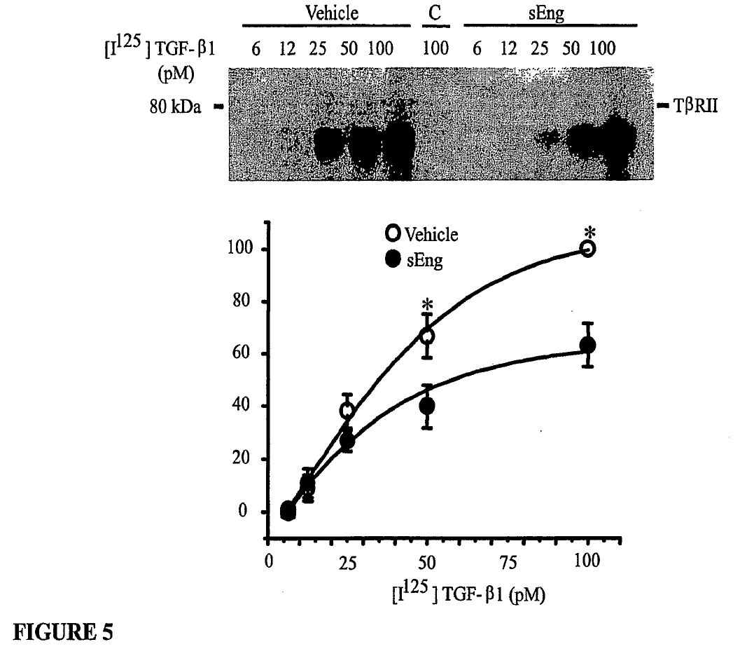

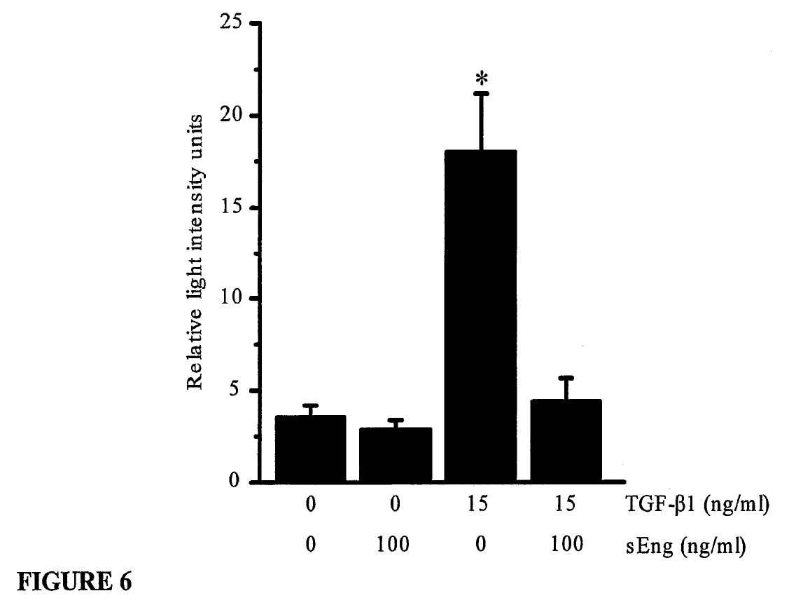

Given that endoglin is a co-receptor for TGF-β1 and -β3 isoforms, we hypothesized that soluble endoglin acts by interfering with cell surface receptor binding. Pre-incubating radio-labeled TGF-β1 with recombinant soluble endoglin significantly reduced its binding to TGF-β receptor type II (TβRII) at both 50 and 100 pM (FIG. 5). Thus, soluble endoglin competes for TGF-β1 binding to its receptors on endothelial cells. To test whether this leads to impaired signaling, the activity of a CAGA-Luc reporter construct was assessed in human endothelial cells. TGF-β1 induced the activation of the Smad 2/3-dependent CAGA-Luc reporter and this response was abolished by treatment with soluble endoglin (FIG. 6).

### Example 3

### Soluble Endoglin is an Anti-Angiogenic Molecule and Induces Vascular Dysfunction

To assess the hemodynamic effects of soluble endoglin, a series of microvascular reactivity experiments in rat renal microvessels were performed.

The methods used for these experiments are described in detail in U.S. Patent Application Publication No. 2006/0067937 and PCT Publication No. WO 06/034507, herein incorporated by reference. Briefly, Evans blue avidly binds to albumin and has been used to quantify in vivo permeability in animals and humans (Green et al., J. Lab. Clin. Med. 111, 173-83, 1988). BALB/c mice were injected intravenously with 1×10⁸ pfu of adenovirus expressing Fc (Control), sEng, sFlt1 or sFlt1+sEng and microvascular permeability measured 48 hours later. One hundred μl of 2% Evans blue dye (in PBS) was injected intravenously. Forty minutes later, mice were perfused via heart puncture with phosphate buffered saline (PBS) containing 2 mM EDTA for 20 minutes. Organs (lung, liver, kidney) were harvested and incubated in formamide. Evans blue dye was extracted by incubating the samples at 70° C. for 24 hours, and its concentration estimated from absorbance at 620 nm and 740 nm. The following formula was used to correct optical densities (O.D) for contamination with heme pigments: O.D. 620 (corrected)=O.D. 620-(1.326×O.D740+0.030).

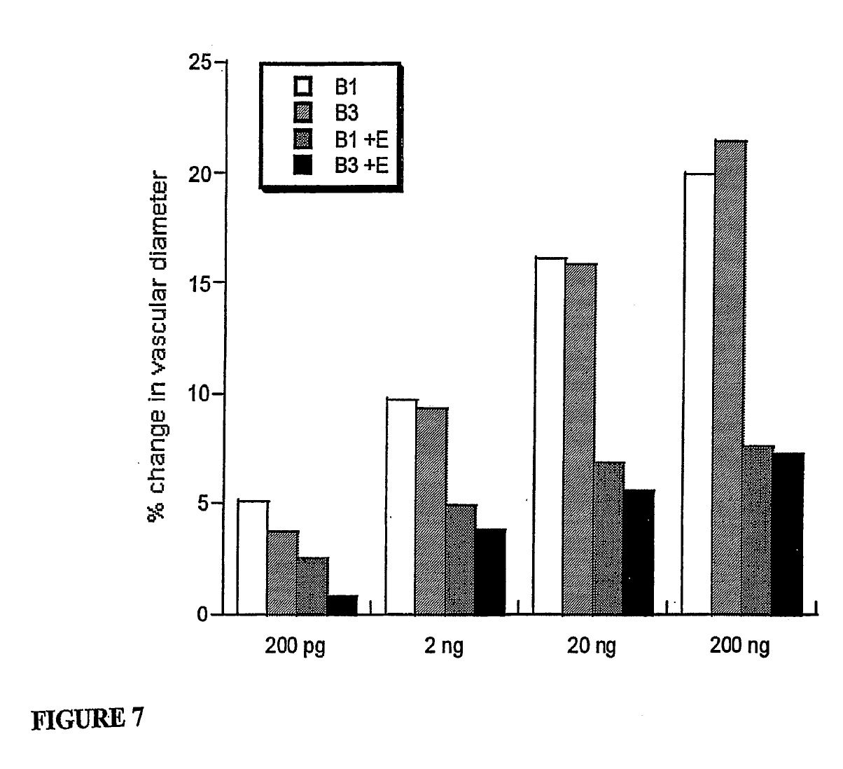

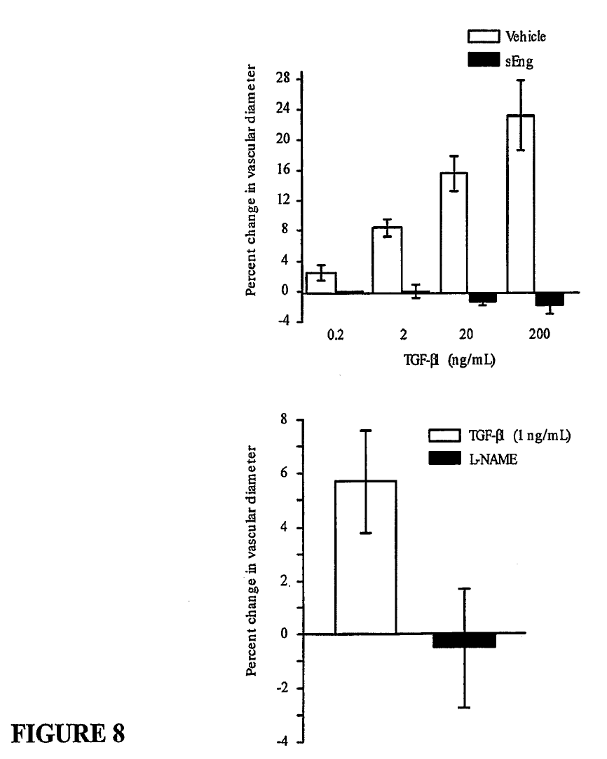

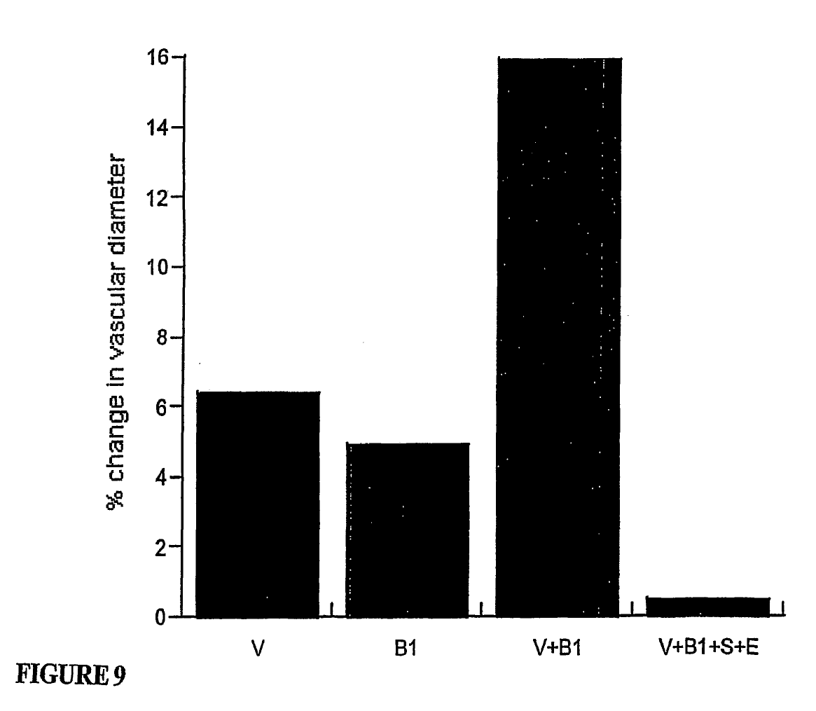

We studied first the effects of TGF-β1 and TGF-β3. Both TGF-β1 and TGF-β3 induced a dose-dependent increase in vascular diameter. Both TGF-β1 and TGF-β3 β3 induced a dose-dependent increase in arterial diameter, whereas TGF-β2, which is not a ligand for endoglin, failed to produce any significant vasodilation (<2% at 0.1 and 1 μg/ml). Importantly in the presence of excess soluble endoglin, the effect of both TGF-β1 and TGF-β3 were significantly attenuated (FIG. 7). This acute effect of TGF-β1 I and TGF-β3 isoforms on vascular tone was also seen in mesenteric vessels (FIG. 8). Finally, the combination of VEGF and TGF-β1 induced vasodilation which was blocked by excess soluble endoglin and sFlt1 (FIG. 9). This suggests that the sFlt1 and soluble endoglin may oppose the physiological vasodilation induced by angiogenic growth factors such as VEGF and TGF-β1 and induce hypertension.

### Example 4

### Model Assay for Angiogenesis

An endothelial tube assay can be used an in vitro model of angiogenesis. Growth factor reduced Matrigel (7 mg/mL, Collaborative Biomedical Products, Bedford, Mass.) is placed in wells (100 μl/well) of a pre-chilled 48-well cell culture plate and is incubated at 37° C. for 25-30 minutes to allow polymerization. Human umbilical vein endothelial cells (30,000+ in 300 μl of endothelial basal medium with no serum, Clonetics, Walkersville, Md.) at passages 3-5 are treated with 10% patient serum, plated onto the Matrigel coated wells, and are incubated at 37° C. for 12-16 hours. Tube formation is then assessed through an inverted phase contrast microscope at 4× (Nikon Corporation, Tokyo, Japan) and is analyzed (tube area and total length) using the Simple PCI imaging analysis software.

### Example 5

### Animal Experiments to Demonstrate the Anti-Tumor Effects of Soluble Endoglin

A subcutaneous tumor model using 786-O renal carcinoma cells (RCC) in nude mice (nu/nu) was used. Briefly, 5 million RCC cells were injected subcutaneously and allowed to grow till they reached a tumor volume of approximately 250 mm³. These mice were then randomized to receive adenovirus expressing soluble endoglin (treated) or control adenovirus expressing CMV vector (control) intravenously (day 0). Tumor growth in these mice was then followed every 3-4 days by measuring tumor volume. In these preliminary data, animal receiving soluble endoglin appeared to have a slower tumor growth.

TABLE 1
Tumor growth in soluble endoglin treated mice.

| Treatment | Tumor volume | Tumor volume | Tumor volume | Tumor volume |
|---|---|---|---|---|
| groups | day 0 | day 4 | day 7 | day 11 |
| Control (n = 4) | 222.2 ± 51.05 | 701 ± 266.31 | 913.25 ± 246.05 | 1031.75 ± 303.37 |
| Treated (n = 3) | 348.66 ± 136.38 | 540.6 ± 80.86 | 773 ± 180.28 | 643.60 ± 182.49 |

* All tumor volumes shown in mm³

### OTHER EMBODIMENTS

The description of the specific embodiments of the invention is presented for the purposes of illustration. It is not intended to be exhaustive or to limit the scope of the invention to the specific forms described herein. Although the invention has been described with reference to several embodiments, it will be understood by one of ordinary skill in the art that various modifications can be made without departing from the spirit and the scope of the invention, as set forth in the claims. All patents, patent applications, and publications referenced herein, including U.S. provisional application No. 60/809,462, are hereby incorporated by reference. Other embodiments are in the claims.

What is claimed is:

1-4. (canceled)

5. A method of inhibiting TGF-β biological activity, comprising contacting said cell with a soluble endoglin compound, or biologically active fragments, derivatives, or analogs thereof, in an amount effective to inhibit said biological activity of TGF-β in said cell.

6. The method of claim 5, wherein said TGF-β is TGF-β1 or TGF-β3.

7. The method of claim 5, wherein said soluble endoglin compound is a soluble endoglin polypeptide comprising a sequence substantially identical to any one of the following sequences: the sequence set forth in SEQ ID NO: 2, amino acids 1 to 587 of SEQ ID NO: 3, or amino acids 40 to 406 of SEQ ID NO: 3.

8. The method of claim 5, wherein said soluble endoglin compound is a soluble endoglin polypeptide, or biologically active fragment thereof, that binds a TGF-β family member.

9. The method of claim 5, wherein said soluble endoglin compound is a soluble endoglin polypeptide, or a biologically active fragment thereof, that binds to a TGF-β receptor.

10. The method of claim 5, wherein said soluble endoglin compound is a soluble endoglin nucleic acid molecule comprising a sequence that encodes a polypeptide having a sequence substantially identical to SEQ ID NO: 2.

11. The method of claim 6, wherein said soluble endoglin nucleic acid molecule comprises a sequence substantially identical to SEQ ID NO: 1.

12. The method of claim 5, wherein said biological activity of TGF-β is selected from the group consisting of inhibition of TGF-β binding to a TGF-β receptor, inhibition of angiogenic activity, conversion from a pro-angiogenic state to an anti-angiogenic state, and reversal or inhibition of TGF-β induced Smad2/3 transcriptional activation.

13. A method for treating or preventing cancer in a subject in need thereof, said method comprising administering to said subject a soluble endoglin compound, or a biologically active fragment, derivative, or analog thereof, wherein said compound has soluble endoglin biological activity, and wherein said administering is for a time and in an amount sufficient to treat or prevent said cancer in said subject.

14. The method of claim 13, wherein said soluble endoglin compound is a soluble endoglin polypeptide, or biologically active fragment thereof.

15. The method of claim 14, wherein said soluble endoglin polypeptide comprises a sequence substantially identical to any one of the following sequences: the sequence set forth in SEQ ID NO: 2, amino acids 1 to 587 of SEQ ID NO: 3, and amino acids 40 to 406 of SEQ ID NO: 3.

16. (canceled)

17. The method of claim 13, wherein said soluble endoglin compound is a soluble endoglin nucleic acid molecule comprising a sequence that encodes a polypeptide having a sequence substantially identical to SEQ ID NO: 2.

18. The method of claim 17, wherein said soluble endoglin nucleic acid molecule comprises a sequence substantially identical to SEQ ID NO: 1.

19. The method of claim 13, wherein said soluble endoglin biological activity is selected from the group consisting of inhibition of TGF-β binding to a TGF-β receptor, inhibition of angiogenic activity, conversion from a pro-angiogenic state to an anti-angiogenic state, and reversal or inhibition of TGF-β induced Smad2/3 transcriptional activation.

20. The method of claim 13, wherein said cancer is a cancer of the breast, prostate, colon, lung, head and neck, liver, kidney, renal system, or endometrium.

21. The method of claim 13, wherein the cancer is metastatic and said method is used to treat said metastasis.

22. The method of claim 13, wherein the cancer is at risk of becoming metastatic and the method is used to prevent said metastasis.

23. The method of claim 13, wherein said cancer is characterized by angiogenic activity or increased TGF-β levels.

24. The method of claim 13, further comprising administering to said subject an additional cancer therapy selected from the group consisting of surgery, radiation therapy, chemotherapy, immune therapy, differentiating therapy, anti-angiogenic therapy, hormone therapy, and hyperthermia.

25. The method of claim 24, wherein said soluble endoglin compound is administered before said additional cancer therapy.

26. The method of claim 24, wherein said soluble endoglin compound is administered during or after said additional cancer therapy.

27. The method of claim 13, further comprising administering to said subject at least one compound selected from the group consisting of an chemotherapeutic agent, an angiogenesis inhibitor, and an anti-proliferative compound.

28. The method of claim 27, wherein said angiogenesis inhibitor is selected from the group consisting of an anti-angiogenic antibody, an antibody that binds VEGF-A, an antibody that binds a VEGF receptor and blocks VEGF binding, sFlt-1, VEGF trap, avastin, endostatin, angiostatin, restin, tumstatin, TNP-470, 2-methoxyestradiol, thalidomide, a peptide fragment of an anti-angiogenic protein, canstatin, arrestin, a VEGF kinase inhibitor, CPTK787, SFH-1, an anti-angiogenic protein, thrombospondin-1, platelet factor-4, interferon-α, an agent that blocks TIE-1 or TIE-2 signaling, or PIH12 signaling, an agent that blocks an extracellular vascular endothelial (VE) cadherin domain, an antibody that binds to an extracellular VE-cadherin domain, an antibody that blocks TGF-β signaling, tetracycline, penicillamine, vinblastine, cytoxan, edelfosine, tegafur or uracil, curcumin, green tea, genistein, resveratrol, N-acetyl cysteine, captopril, a cyclooxygenase-2 inhibitor, celecoxib, and rofecoxib.

29-45. (canceled)
